(19) United States
(12) Patent Application Publication
(10) Pub. No.: US 2022/0112185 A1
(43) Pub. Date: Apr. 14, 2022

(54) NOVEL PHENYL PROPIONIC ACID DERIVATIVES AND USES THEREOF

(71) Applicant: ILDONG PHARMACEUTICAL CO., LTD., Seoul (KR)

(72) Inventors: Jae-Hoon KANG, Seoul (KR); Hong-Sub LEE, Gyeonggi-do (KR); Kyung-Mi AN, Gunpo-si (KR); Chang-Hee HONG, Seoul (KR); Hyun-Jung KWAK, Suwon-si (KR); Shuo-Lin CUI, Hwaseong-si (KR); Hyo-Jung SONG, Seoul (KR)

(73) Assignee: ILDONG PHARMACEUTICAL CO., LTD., Seoul (KR)

(21) Appl. No.: 17/559,546

(22) Filed: Dec. 22, 2021

Related U.S. Application Data
(63) Continuation-in-part of application No. 17/523,652, filed on Nov. 10, 2021.
Application No. 17/523,652 is a continuation of application No. 16/467,654, filed on Jun. 7, 2019, now Pat. No. 11,225,472.

(30) Foreign Application Priority Data
Dec. 15, 2016 (KR) 10-2016-0171541
Dec. 13, 2017 (KR) 10-2017-0171228

(51) Int. Cl. C07D 417/12; C07D 409/12; C07D 405/14

(52) U.S. Cl. CPC C07D 417/12; C07D 405/14; C07D 409/12

(57) ABSTRACT

Compounds of Formula (I), racemates, enantiomers, diastereomers thereof or pharmaceutical acceptable salts thereof, or pharmaceutical compositions containing the compounds, racemates, enantiomers, diastereomers thereof, and a manufacturing method thereof are disclosed. These compounds have GPR40 agonist activity and are capable of modulating blood glucose levels and glucose-dependent insulin secretion mechanism, and, thus, exhibit excellent glucose lowering efficacy without the risk of hypoglycemia. These compounds could be used in preventing and/or treating type 2 diabetes through adequate control of blood glucose.

### CROSS-REFERENCE TO RELATED APPLICATIONS

This application is a continuation-in-part of U.S. application Ser. No. 17/523,652 filed Nov. 10, 2021, which is a continuation of U.S. application Ser. No. 16/467,654 filed Jun. 7, 2019 (allowed), which is the U.S. national phase application filed under 35 U.S.C. § 371 claiming benefit to International Patent Application No. PCT/KR2017/014757, filed on Dec. 14, 2017, which is entitled to priority under to Korean Patent Application No. 10-2016-0171541, filed Dec. 15, 2016 and Korean Patent Application No. 10-2017-0171228, filed Dec. 13, 2017, the disclosures of which are incorporated herein by reference in their entireties.

### TECHNICAL FIELD

The present invention relates to phenyl propionic acid derivatives, isomers, and pharmaceutically permissible salts thereof, and on its medicinal uses.

### BACKGROUND ART

Diabetes mellitus (DM) is mainly divided into Type 1 and Type 2 diabetes. Type 1 diabetes mellitus (T1DM) is a condition characterized by the genetically predisposed destruction of pancreatic β-cells that are responsible for the production of insulin, which results in the body's inability to produce sufficient insulin for the control of blood glucose level. Type 2 diabetes mellitus (T2DM), covering up to 95% of the total diabetic patients, is an acquired disease in which environmental factors cause somatic cells to become insulin-resistant, which in turn disables effective absorption of blood glucose. Chronic rise in blood glucose level caused by insulin abnormality leads to serious complications, including obesity, neuralgia, diabetic retinopathy, nephropathy, cardiovascular diseases and dyslipidemia.

Early symptoms of onset of the disease include hyperuresis and unidentified weight loss, and the disease itself can be properly diagnosed through precise examinations of HbA1c level, fasting and postprandial glucose level, and glucose tolerance test. T2DM patients generally display HbA1c level of over 6.5%, fasting plasma glucose (FPG after 8 hrs) level of over 126 mg/dL, and the postprandial level (2 hrs-Plasma Glucose) of over 200 mg/dL.

According to data from the International Diabetes Federation (IDF), the number of T2DM patients around the globe increased from 30 million in 1985 to 415 million in 2015, and is expected to rise by 7 million annually to 642 million adult patients by 2040, which marks over 10% of the global population. In addition, approximately 50% of the patients also suffer from related complications with 5 million resulting deaths, making them responsible for 14.5% of the global death count.

Increasing number of patients has resulted in subsequent growth of the global market for the treatment of T2DM. The market value increased significantly from 28.8 billion dollars in 2009 to 63.6 billion dollars in 2014 and is expected to reach 163.2 billion dollars by 2020. Dietary habits, lack of exercise and irregular lifestyle have been pointed out as the indirect causes of such increase in the occurrence of diabetes mellitus. Therefore, patients are prescribed a variety of medicinal treatments along with balanced diet, regular exercise and maintenance of healthy weight, but there still are unmet needs for discovery of novel medications for the full recovery of the disease.

Currently being actively prescribed medications for T2DM can be categorized based on their mechanisms of action. However, each type has shortcomings which cannot be overcome. For example, Metformin of biguanide type, the primary treatment for T2DM, places patients at risk of diarrhea, abdominalgia, dyspepsia, and lack of durability in long-term use. Sulfonylureas (SUs), independent from blood glucose level, stimulate pancreatic β-cells and thus place patients at risk of hypoglycemia. Liver safety concerns, cardiovascular (CV) risk, weight gain and risk of bladder cancer have been reported with thiazolidinediones, which led to withdrawal of the drug from the market. Sodium-glucose co-transporter-2 (SGLT-2) inhibitors make patients become vulnerable to urinary tract and genital infections, and α-glucosidase inhibitors may induce side-effects including dyspepsia and diarrhea. Furthermore, Dipeptidyl peptidase-4 (DPP-IV) inhibitors are limited to patients without any renal conditions. Therefore, there is a need for discovery of novel medications for T2DM which is able to overcome such limitations, and accordingly, GPR40 (G-protein-coupled receptor 40) agonists have recently been gaining attention.

GPR40, a seven-transmembrane protein, is a type of G-protein-coupled receptors (GPCRs) of the rhodopsin family, and is primarily expressed in β-cells of pancreatic islets. Since its primary ligands are medium-to-long change fatty acids, the receptor is also known as Free fatty acid receptor 1 (FFAR1).

The mechanism of insulin secretion from pancreatic β-cell's through GPR40 is mainly determined by either endogenous ligands or synthetic GPR40 agonists that bind to the receptor. To date, the predominant view holds that GPR40 signals primarily through the heterotrimeric G-protein G_{q/11}. It typically signals through Phospholipase C (PLC)-mediated hydrolysis of membrane phospholipids and the generation of Inositol 1,4,5-trisphosphate (IP₃) and Diacylglycerol (DAG). As downstream second messenger molecules, it subsequently activates Protein Kinase D1 (PKD1) to induce F-actin protein modification, and calcium ion secretion to ultimately induce insulin secretion.

The mechanism that GPR40 activation induces insulin secretion with blood glucose-dependent manner was proven through experiments using rodent models. (Diabetes, 2007, 56, 1087-1094; Diabetes, 2009, 58, 1067-1076). Such blood glucose-dependent mechanism of insulin secretion has no risk of hypothermia, which makes GPR40 an attractive target for novel drug development. In addition, GPR40 plays a role in maintaining pancreatic β-cell survival by alleviating chronic inflammation through regulation of PDX-1 and BCL2, and by mitigating β-Cell lipotoxicity-induced metabolic inflammation damage through Inhibiting the TLR4-NF-κB pathway (BMC Cell Biol., 2014, 15, 24; J Diabetes Res., 2019, 5487962). Since inflammation-mediated P cell death is known to decrease β-cell mass in diabetes leading to reduced insulin secretion, GPR40 agonists will maintain optimal glycemic control during long-term treatment. Furthermore, since the distribution of GPR40 expression is relatively limited, there is low risk of adverse effects in other organs, and improvement of blood-glucose homeostasis through GPR40 activation could potentially be beneficial for other metabolic disorders including obesity and hypertension.

Based on these advantages, the pharmaceutical industry has made efforts to develop GPR40 agonists over the past few decades, but no drugs are on the market. Fasiglifam, developed by Takeda, is the first GPR40 agonist to enter Phase III clinical evaluation. Although fasiglifam showed hypoglycemic effect in T2DM patients in the Phase II clinical trials, it was withdrawn from the Phase III clinical trials due to concerns of liver safety (Diabetes obes metab., 2015, 17, 675-681).

It is definitive that discovery of novel GPR40 agonists which bear mechanism of glucose-dependent insulin secretion is in necessity of modern society, where the number of patients suffering from metabolic disorders including T2DM is still drastically increasing, to provide effective means of treating such metabolic diseases.

### DISCLOSURE

### Technical Problem

The objective of the present invention relies on providing agonists acting on GPR40; particularly novel phenyl propionic acid derivatives, isomers, and pharmaceutically available salts thereof.

In addition, the object of the present invention is to provide medicinal use for treatment of GPR40-mediated disorders.

However, the technical object to be achieved in the present invention is not limited to those aforementioned above, and other objects may be clearly understood by those skilled in the art from the following description.

### Technical Solution

Compounds represented by Formula (I); a racemate, an enantiomer, or a diastereomer thereof, or a pharmaceutically acceptable salt thereof:

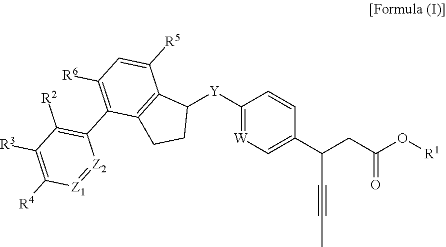

R¹ is hydrogen, or C₁₋₄ linear or branched alkyl;

R² is hydrogen, cyano, hydroxyl, C₁₋₄ linear or branched alkyl, or C₁₋₄ linear or branched alkoxy;

R³ and R⁴ are each independently hydrogen, halogen, cyano, C₁₋₄ linear or branched alkoxy, or OR⁸;

wherein R⁸ is hydrogen; C₃₋₁₀ heterocycloalkyl comprising 1-4 hetero atoms selected from the group consisting of N, O, and S; or substituted alkyl with C₃₋₁₀ heterocycloalkyl comprising 1-4 hetero atoms selected from the group consisting of N, O, and S;

R⁵ and R⁶ are each independently hydrogen, halogen, cyano, halomethyl, hydroxyl, C₁₋₄ linear or branched alkyl, or C₁₋₄ linear or branched alkoxy;

Y is NH or O;

Z₁, Z₂ and W are each independently CR⁷ or N;

wherein R⁷ is hydrogen, halogen, cyano, hydroxyl, C₁₋₄ linear or branched alkyl, or C₁₋₄ linear or branched alkoxy.

This invention is provided to the compounds according to Formula (I), as racemates, enantiomers, diastereomers thereof; or pharmaceutical acceptable salts, for the treatment of disorders; wherein responsive to agonism of the GPR40.

This invention is provided to the process of compounds according to Formula (I), as racemates, enantiomers, diastereomers thereof; or pharmaceutical acceptable salts.

### Advantageous Effects

The compounds of the present invention, as GPR40 agonists, are orally available and are extremely effective in lowering blood glucose level to normal state without any risk of inducing hypoglycemia via glucose-dependent insulin secretion. Therefore, compounds and/or therapeutically effective pharmaceutical composition comprising the compounds of the present invention are useful in the treatment, delaying, and/or regression of symptoms of type 2 diabetes. In addition, compounds of the present invention modulate glucose excursion via GPR40 activation; the therapeutic effect can also be potentially available in obesity and hypertension.

In addition, since the compounds of the present invention have shown improved and/or enhanced therapeutic effects of alleviating and/or treating symptoms of type 2 diabetes compared to pre-existing medications when evaluated of glucose-lowering effects of the compounds on animal models and/or human-organ derived materials, the compounds can be evaluated as being highly useful to potential beneficiaries of the present invention.

### [Modes]

Compounds represented by Formula (I); a racemate, an enantiomer, or a diastereomer thereof, or a pharmaceutically acceptable salt thereof:

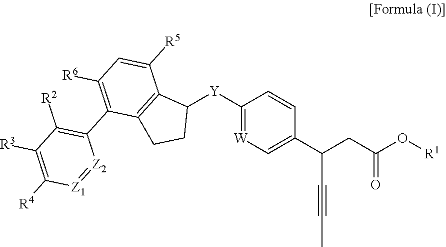

R¹ is hydrogen, or C₁₋₄ linear or branched alkyl;

R² is hydrogen, cyano, hydroxyl, C₁₋₄ linear or branched alkyl, or C₁₋₄ linear or branched alkoxy;

R³ and R⁴ are each independently hydrogen, halogen, cyano, C₁₋₄ linear or branched alkoxy, or OR⁸;

wherein R⁸ is hydrogen; C₃₋₁₀ heterocycloalkyl comprising 1-4 hetero atoms selected from the group consisting of N, O, and S; or substituted alkyl with C₃₋₁₀ heterocycloalkyl comprising 1-4 hetero atoms selected from the group consisting of N, O, and S;

R⁵ and R⁶ are each independently hydrogen, halogen, cyano, halomethyl, hydroxyl, C₁₋₄ linear or branched alkyl, or C₁₋₄ linear or branched alkoxy;

Y is NH or O;

Z₁, Z₂ and W are each independently CR⁷ or N;

wherein R⁷ is hydrogen, halogen, cyano, hydroxyl, C₁₋₄ linear or branched alkyl, or C₁₋₄ linear or branched alkoxy.

Examples of preferred compounds according to the Formula (I) in present invention are following:
(S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid;
(S)-3-(4-(((R)-7-fluoro-4-(6-((3-methyloxetan-3-yl)methoxy)pyridin-3-yl)-2,3-dihydro-1H-inden-1-yl)oxy)phenyl)hex-4-ynoic acid;
(S)-3-(4-(((R)-4-(6-(2-(1,1-dioxidothiomorpholino)ethoxy)pyridin-3-yl)-7-fluoro-2,3-dihydro-1H-inden-1-yl)oxy)phenyl)hex-4-ynoic acid;
(S)-3-(4-(((R)-7-fluoro-4-(6-(oxetan-3-yloxy)pyridin-3-yl)-2,3-dihydro-1H-inden-1-yl)oxy)phenyl)hex-4-ynoic acid;
(S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-7-fluoro-4-(6-((tetrahydro-2H-pyran-4-yl)oxy)pyridin-3-yl)-2,3-dihydro-1H-inden-1-yl)oxy)phenyl)hex-4-ynoic acid;
(S)-3-(4-(((R)-7-fluoro-4-(6-(((S)-tetrahydrofuran-3-yl)oxy)pyridin-3-yl)-2,3-dihydro-1H-inden-1-yl)oxy)phenyl)hex-4-ynoic acid;
(S)-3-(4-(((R)-7-fluoro-4-(4-methyl-6-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-7-fluoro-4-(2-methyl-6-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-4-(5-chloro-6-(((R)-tetrahydrofuran-3-yl)oxy)pyridin-3-yl)-7-fluoro-2,3-dihydro-1H-inden-1-yl)oxy)phenyl)hex-4-ynoic acid;
(S)-3-(4-(((R)-7-fluoro-4-(5-(((R)-tetrahydrofuran-3-yl)oxy)pyridin-2-yl)-2,3-dihydro-1H-inden-1-yl)oxy)phenyl)hex-4-ynoic acid;
(S)-3-(4-(((R)-7-fluoro-4-(4-methyl-6-((3-methyloxetan-3-yl)methoxy)pyridin-3-yl)-2,3-dihydro-1H-inden-1-yl)oxy)phenyl)hex-4-ynoic acid;
(S)-3-(4-(((R)-7-fluoro-4-(2-methyl-6-((3-methyloxetan-3-yl)methoxy)pyridin-3-yl)-2,3-dihydro-1H-inden-1-yl)oxy)phenyl)hex-4-ynoic acid;
(S)-3-(4-(((R)-7-fluoro-4-(5-((3-methyloxetan-3-yl)methoxy)pyridin-2-yl)-2,3-dihydro-1H-inden-1-yl)oxy)phenyl)hex-4-ynoic acid;
(S)-3-(4-(((R)-7-fluoro-4-(5-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-7-fluoro-4-(5-((tetrahydro-2H-pyran-4-yl)oxy)pyridin-3-yl)-2,3-dihydro-1H-inden-1-yl)oxy)phenyl)hex-4-ynoic acid;
(S)-3-(4-(((R)-4-(5-chloro-6-((tetrahydro-2H-pyran-4-yl)oxy)pyridin-3-yl)-7-fluoro-2,3-dihydro-1H-inden-1-yl)oxy)phenyl)hex-4-ynoic acid;
(S)-3-(4-(((R)-4-(5-cyano-6-(((R)-tetrahydrofuran-3-yl)oxy)pyridin-3-yl)-7-fluoro-2,3-dihydro-1H-inden-1-yl)oxy)phenyl)hex-4-ynoic acid;
(S)-3-(4-(((R)-4-(5-cyano-6-((tetrahydro-2H-pyran-4-yl)oxy)pyridin-3-yl)-7-fluoro-2,3-dihydro-1H-inden-1-yl)oxy)phenyl)hex-4-ynoic acid;
(S)-3-(4-(((R)-5-cyano-4-(6-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-5-fluoro-4-(6-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-5-methoxy-4-(6-(((R)-tetrahydrofuran-3-yl)oxy)pyridin-3-yl)-2,3-dihydro-1H-inden-1-yl)oxy)phenyl)hex-4-ynoic acid;
(S)-3-(4-(((R)-5-cyano-4-(6-((tetrahydro-2H-pyran-4-yl)oxy)pyridin-3-yl)-2,3-dihydro-1H-inden-1-yl)oxy)phenyl)hex-4-ynoic acid;
(S)-3-(4-(((R)-5-fluoro-4-(6-((tetrahydro-2H-pyran-4-yl)oxy)pyridin-3-yl)-2,3-dihydro-1 H-inden-1-yl)oxy)phenyl)hex-4-ynoic acid;
(S)-3-(4-(((R)-5-methoxy-4-(6-((tetrahydro-2H-pyran-4-yl)oxy)pyridin-3-yl)-2,3-dihydro-1H-inden-1-yl)oxy)phenyl)hex-4-ynoic acid;
(S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid;
3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid.

In the exemplary embodiments, “heterocycloalkyl” refers to cycloalkyl groups containing hetero atoms. Exemplary heterocycloalkyl groups include, but not limited to, oxetane, tetrahydrofuran, pyran, azetidine, pyrrolidinyl, piperazinyl, morpholine or thiomorpholine.

In the exemplary embodiments, “C₁₋₄ alkyl” is a saturated hydrocarbon group with linear or branched chains of 1 to 4 carbon atoms. Exemplary alkyl groups include, but not limited to, methyl, ethyl, propyl, butyl, 1-methylethyl, diethyl or dimethyl.

In the exemplary embodiments, “C₁₋₄ alkoxy” is an OR group with 1 to 4 carbon atoms and R is an alkyl as defined above. Exemplary C₁₋₄ alkoxy groups include, but not limited to, methoxy, ethoxy, propoxy, butoxy, 1-methylethoxy or 1,1-dimethylethoxy.

In the exemplary embodiments, “C₁₋₂ alkyl” is a saturated hydrocarbon group with linear or branched chains of 1 to 2 carbon atoms. Exemplary alkyl groups include, but not limited to, methyl or ethyl.

In the exemplary embodiments, “C₁₋₂ alkoxy” is an OR group with 1 to 2 carbon atoms and R as defined above. Exemplary C₁₋₂ alkoxy groups include, but not limited to, methoxy or ethoxy.

In the exemplary embodiments, “halo” is defined as bromine, fluorine, or chlorine atom.

Herein, the term “pharmaceutically acceptable” refers to a usable component or composition, within the medical criteria, that does not incorporate irrational risk of toxicity.

The compounds of the embodiments contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. A stereoisomer is referred to as an enantiomer, and a mixture of such isomers is often called an enantiomeric mixture. A 50:50 mixture of enantiomers is referred to as a racemic-mixture or a racemate.

In the exemplary embodiments, “diastereomer” refers to a stereoisomer with two or more centers of chirality and whose molecules are not mirror images of one another. Diastereomers have different physical properties, such as melting points, boiling points, spectral properties, and reactivity. Mixtures of diastereomers may become separated under high resolution analytical procedures such as electrophoresis and chromatography.

In the exemplary embodiments, “enantiomers” refer to two stereoisomers of a compound which are non-superimposable mirror images of one another.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the exemplary embodiments is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or methanesulfonic acid, or the like.

If the compound of the exemplary embodiments is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminium and lithium.

In another aspect, the present invention provides a method of preparing the compounds represented by Formula (I) or pharmaceutically approved salts thereof.

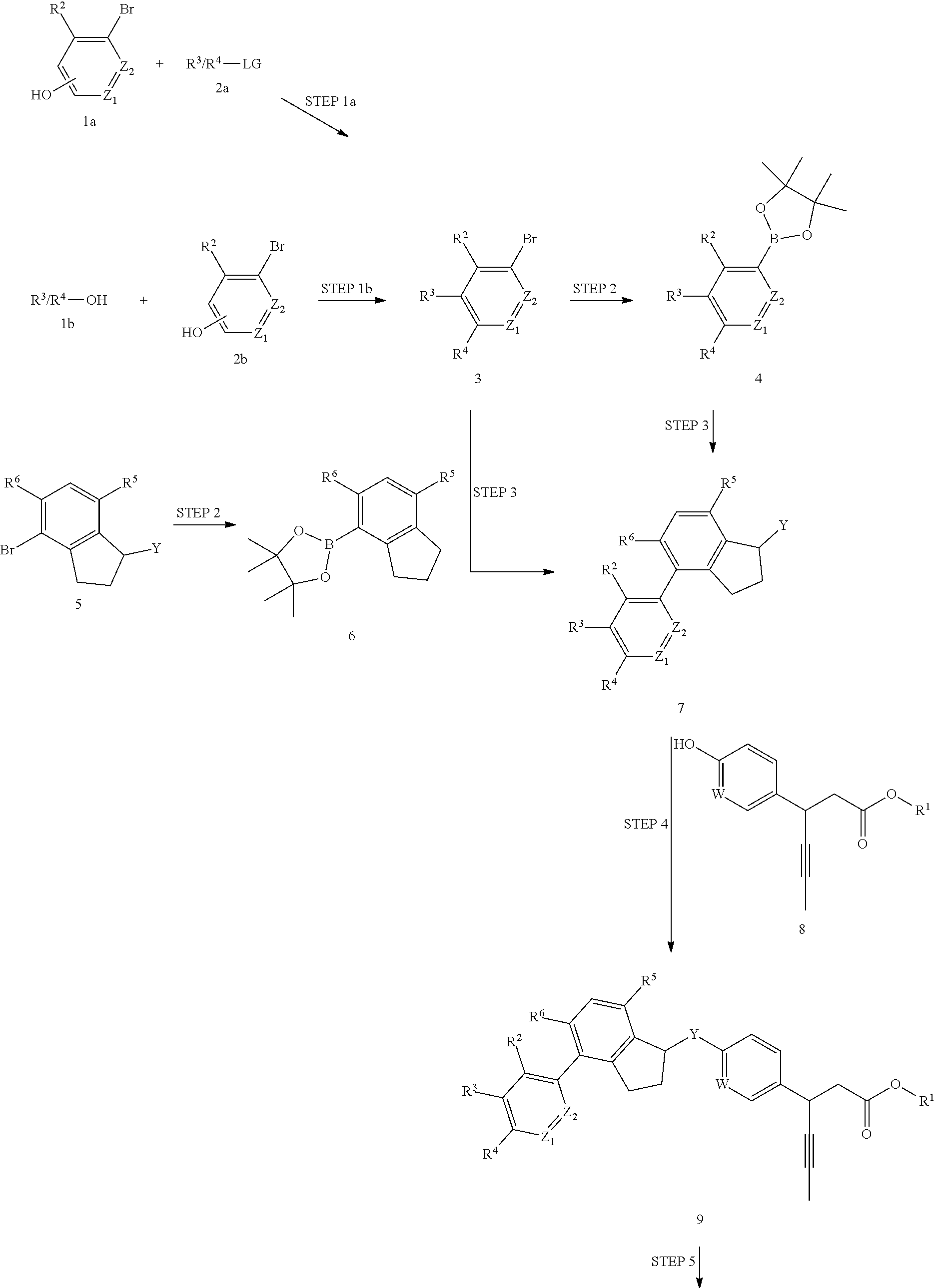

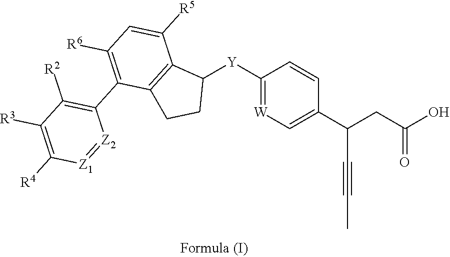

Unless otherwise stated, the groups, residues, and substituent, particularly R⁶, Y, W, Z₁, and Z₂ are defined as above and hereinafter.

R¹ is hydrogen, or C₁₋₄ linear or branched alkyl;

R² is hydrogen, cyano, hydroxyl, C₁₋₄ linear or branched alkyl, or C₁₋₄ linear or branched alkoxy;

R³ and R⁴ are each independently hydrogen, halogen, cyanide, C₁₋₄ linear or branched alkoxy, or OR⁸;

wherein R⁸ is hydrogen; C₃₋₁₀ heterocycloalkyl comprising 1-4 hetero atoms selected from the group consisting of N, O, and S; or substituted alkyl with C₃₋₁₀ heterocycloalkyl comprising 1-4 hetero atoms selected from the group consisting of N, O, and S;

R⁵ and R⁶ are each independently hydrogen, halogen, cyano, halomethyl, hydroxyl, C₁₋₄ linear or branched alkyl, or C₁₋₄ linear or branched alkoxy;

Y is NH or O;

Z₁, Z₂ and W are each independently CR⁷ or N;

wherein R⁷ is hydrogen, halogen, cyano, hydroxy, C₁₋₄ linear or branched alkyl, or C₁₋₄ linear or branched alkoxy.

Specifically, the process of preparing the compounds of Formula (I) includes;

The step of preparing compound 3 through substitution reaction of compound 1 and compound 2 (Step 1);

The step of preparing a compound that is represented either by compound 4 or compound 6 through boronylation reaction of compound 4 and compound 6 (Step 2);

The step of preparing compound 7 through Suzuki coupling reactions of compound 3 or compound 5 and compound 4 or compound 6 (Step 3);

The step of preparing compound 9 through Mitsunobu condensation of compound 7 and compound 8 (Step 4); or

The step of preparing compounds of Formula (I) through hydrolysis reaction of compound 9 (Step 5).

The preparing processes of Formula (I) can be described in more detail for each step as follows;

i) In Step 1, compound 3 can be prepared through substitution of leaving group of compound 2 with compound 1. In addition, solvents available for the reaction include N,N-dimethylformamide, acetonitrile, dimethyl sulfoxide or toluene, and bases used in the reaction include cesium carbonate, potassium carbonate or sodium hydride. To be specific, the step describes preparation of compound 3 through substitution reaction of compound 1 and compound 2 with adequate solvents and bases, for example, N,N-dimethylformamide and potassium carbonate.

ii) The Step 2 describes the process of preparing compound 4 or compound 6 through boronylation reaction of compound 3 or compound 5 with equivalent or excessive use of boronylation reagents and metal catalysts. Metal catalysts, more specifically palladium catalysts, include [1,1′-Bis(diphenylphosphino)ferrocene]dichloro Palladium (II), dicholoromethane (Pd(dppf)Cl₂.DCM) or Palladium tetrakis(triphenylphosphine) (Pd(PPh₃)₄). In addition, solvents involved in the reaction include dichloromethane, acetonitrile, 1,4-dioxane or toluene. The boronic-reagent can be selected from either Bis(pinacolato)diboron or Bis(neopentylglycolato)diboron. To be more specific, the process of preparing compound 4 can be described in the following reaction; A reaction of a solution of compound 3 and adequate catalysts, boronic-reagents, base and solvents, for example, 1,4-dioxane with Pd(dppf)Cl₂, potassium acetate and Bis(pinacolato)diboron.

iii) The Step 3 describes the process of preparing compounds that are represented by compound 7 from Suzuki coupling reaction of compound 4 or compound 6 obtained from Step 2 and compound 3 or compound 5. The coupling reaction can be processed with adequate combinations of palladium catalysts and bases, and the catalysts available for the reaction include Tetrakis(triphenylphosphine) (Pd(PPh₃)₄), Bis(triphenylphosphine)Palladium (II) dichloride (PdCl₂(PPh₃)₂), Palladium dichloride (PdCl₂) or Palladium acetate (Pd(OCOCH₃)₂). In addition, solvents used for the reaction include tetrahydrofuran, diethylether, diphenylether, diisopropylether, N,N-dimethylformamide, dimethylacetamide, dimethylsulfoxide, dichloromethane, chlorobenzene, toluene, benzene or water or mixture of these solvents. To be more specific, the Step describes the process of preparing compound 7 through Suzuki coupling reaction of compound 3 and compound 4 with the combination of adequate solvents, catalyst, ligand and base, for example, mixture of toluene and water with Pd₂(dba)₃, biphenyl-dicyclohexyl-phosphine, and potassium phosphate tribasic.

iv) The Step 4 describes the process of preparing compound 9 through Mitsunobu reaction of compound 7 and compound 8. To be more specific, compound 9 can be prepared from Mitsunobu reaction of the mixture solution of compound 7 and compound 8 with triphenylphosphine and 1,1′-(azodicarbonyl)dipiperidine (ADDP) under 0° C.

v) The Step 5 describes the process of preparing compounds of Formula (I) through hydrolysis reaction of compound 9 under basic condition. In particular, compounds of Formula (I) can be prepared through compound 9 reacting with adequate base under room temperature, resulting the reduction of ester to carboxylic acid. Bases available for the reaction include potassium hydroxide, sodium hydroxide or lithium hydroxide. To be more specific, compounds of Formula (I) can be prepared from the reaction of compound 9 with adequate base, for example, lithium hydroxide.

The present invention provides a pharmaceutical composition for the treatment of metabolic disorders, which comprises the compounds of Formula (I), racemate, enantiomer, diastereomer thereof, or pharmaceutically acceptable salts thereof. Compounds of the invention intended for pharmaceutical use comprise compounds of Formula (I), their pharmaceutically acceptable salts, solutions, and hydrates.

The term “prevention”, as used herein, covers any inhibition or regression of diseases that are induced by the compounds of the present invention.

The term “treatment”, as used herein, covers any treatment of diseases in a mammal, particularly a human, and includes inhibiting the diseases, i.e., arresting the development; or relieving the diseases, i.e., inducing regression of the diseases and/or their symptoms or conditions and slowing disease progression.

The term “metabolic disorder”, as used herein, refers to any disorders caused by metabolic abnormality in lipids or glucose, and includes, but not limited to, obesity, type 2 diabetes, disturbed glucose tolerance, insulin resistance, hyperglycemia, hyperlipidemia, hypertriglyceridemia, hypercholesteremia, and dyslipidemia.

An aspect of the present invention provides a method of treating metabolic disorders in a subject in need thereof, comprising administration of effective amounts of the pharmaceutical composition to the subject. The dosage of the pharmaceutical composition may vary depending on the patient's weight, age, gender, physical condition, diet, time and mode of administration, excretion rates, and severity of illness, but is readily apparent to those skilled in the art. Mammals, preferably humans, are desirable for the individuals without limit.

The term “therapeutically effective amount” refers to an amount of a compound disclosed herein that ameliorates, attenuates or eliminates a particular disease or condition or prevents or delays the onset of a particular disease or condition. In the case of diabetes mellitus, the therapeutically effective amount of the drug may reduce postprandial blood glucose level; reduce HbA1c level; treat or inhibit diabetic retinopathy or nephropathy; inhibit (slow to some extent and preferably stop) progress of diabetes; weight loss; ameliorate or enhance pancreatic β-cell function; and/or relieve to some extent one or more of the symptoms associated with diabetes. To the extent the drug may modulate blood glucose level to normal state.

The “pharmaceutical composition” as used herein may contain effective component and pharmaceutically acceptable formulation, and the pharmaceutical compositions suitable for the delivery of compounds and methods for their preparation can be readily apparent to those skilled in the art.

The pharmaceutical composition as used herein can be administered either orally or parenterally through diverse formulations, and the effective dose of administration may vary depending on physical condition, body weight and severity of the illness of the subject, formulation, and administration time and route, but can be readily determined by those skilled in the art.

Formulations suitable for oral administration include tablets, pills, solid/soft capsules, liquid, suspension, emulsifier, syrups, granules, and elixir, and typically comprise diluents (i.e., lactose, dextrose, sucrose mannitol, sorbitol, cellulose, and/or glycine) and lubricant (i.e., silica, talc, stearic acid and its magnesium or calcium salt and/or polyethylene glycol). Tablets of the formulation also comprise binders including magnesium aluminium silicate, starch paste, gelatin, methyl cellulose, sodium carboxy methyl cellulose, and/or polyvinyl pyrrolidine, and depending on circumstances, tablets may comprise disintegrants including starch, agar, alginic acid or its sodium salt, or boiling mixture, absorbent, coloring agent, flavoring agents, or sweeteners.

The pharmaceutical composition as used herein is administered with pharmaceutically effective amounts. The term “pharmaceutically effective amount” refers to sufficient amount of a compound that can treat disease with rational and adequate benefit/risk ratio, and the effective amount can be readily determined depending on the types of subject's illness, severity, activity of the compound, sensitivity of the subject to the compound, administration time, route and excretion ratio, treatment interval, factors including co-administered drugs and other well-known medical factors. Compounds according to embodiments of the present invention can be combined or co-administered with other types of drugs as a combination therapy or monotherapy and can be administered with add-on therapy to the pre-existing treatment with single or multiple administration. Aforementioned factors must be all considered adequately to determine within the bounds of goal achieving maximum therapeutic effect with minimum amounts of the compound without harmful or serious adverse effects, and this process can be readily determined by those skilled in the art.

In particular, the therapeutically effective amount of the compound can vary depending on the subject's age, gender, and body weight, and typically ranges from 0.001 to 150 mg per 1 kilogram of body weight, desirably from 0.01 to 100 mg/kg/day or 0.01 to 100 mg/kg/48 hrs with Q.D. (once a day), B.I.D. (twice a day) or T.I.D (three times a day).

Embodiments of the present invention are explained in detail through, but not limited to, the following examples and experimental examples.

[Intermediates]

### <Intermediate 1> (3S)-3-(4-Hydroxy-phenyl)-hex-4-ynoic Acid methyl ester

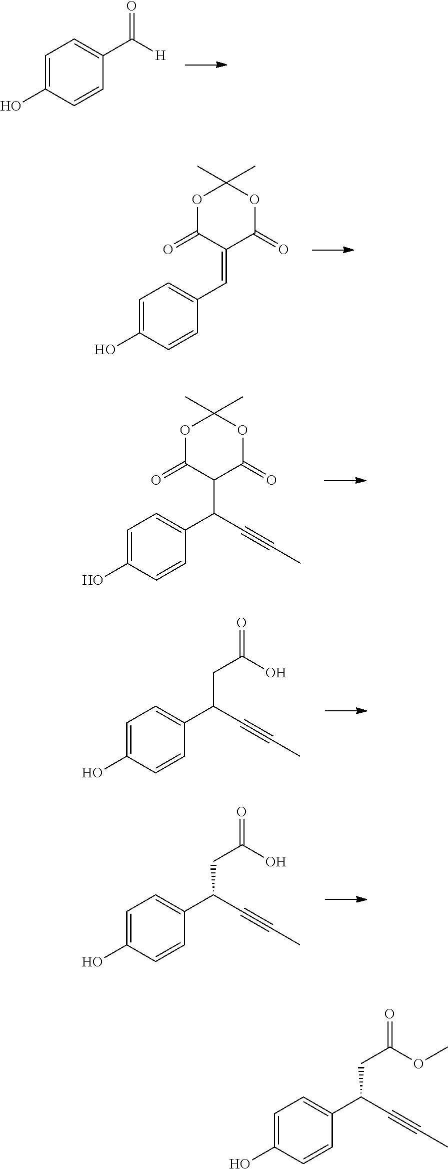 [SMILES: O=Cc1ccc(O)cc1 ; CC1(C)OC(=O)C(=Cc2ccc(O)cc2)C(=O)O1 ; CC#CC(c1ccc(O)cc1)C1C(=O)OC(C)(C)OC1=O ; CC#CC(CC(=O)O)c1ccc(O)cc1 ; CC#C[C@@H](CC(=O)O)c1ccc(O)cc1 ; CC#C[C@@H](CC(=O)OC)c1ccc(O)cc1]

### Step 1: 5-(4-Hydroxy-benzylidene)-2,2-dimethyl-[1,3]dioxane-4,6-dione

4-Hydroxybenzaldehyde (1.0 eq.) was dissolved in water (0.9 M) at 75° C. Subsequently, a solution of Meldrum's acid (1.1 eq.) in water (1.2 M) was added to the reaction mixture. The mixture was stirred at 75° C. for 2 h, and then additional portion of Meldrum's acid (0.5 eq.) in water (1.2 M) was added to the reaction mixture. The mixture was stirred at 75° C. for another 2 h. The mixture was allowed to reach room temperature and filtered with iced water. The wet solid was dried in an oven (50° C.) to afford 5-(4-hydroxy-benzylidene)-2,2-dimethyl-[1,3]dioxane-4,6-dione.

### Step 2: (+/−)-5-[l-(4-Hydroxy-phenyl)-but-2-ynyl]-2,2-dimethyl-[1,3]dioxane-4,6-dione

1-Propynylmagnesium bromide in tetrahydrofuran (0.5 N, 3.0 eq.) was added dropwise to a solution of 5-(4-hydroxy-benzylidene)-2,2-dimethyl-1,3-dioxane-4,6-dione (1.0 eq.) in tetrahydrofuran anhydrous (0.4 M) at 4° C. under N₂ atmosphere. The reaction mixture was stirred at room temperature for 30 min. The mixture was quenched with saturated aqueous ammonium chloride solution and washed with hexane. After the aqueous layer was collected, the mixture was acidified with 1.0 M aqueous hydrochloric acid solution and diluted with ethyl acetate. The organic layer was washed with water and brine, dried over magnesium sulfate, filtered and concentrated. The resultant residue was purified by flash column chromatography on silica gel eluting to afford (+/−)-5-[l-(4-hydroxy-phenyl)-but-2-ynyl]-2,2-dimethyl-[1,3]dioxane-4,6-dione.

### Step 3: (+/−)-3-(4-Hydroxy-phenyl)-hex-4-ynoic Acid

(+/−)-5-[l-(4-Hydroxy-phenyl)-but-2-ynyl]-2,2-dimethyl-[1,3]dioxane-4,6-dione (1.0 eq.) was dissolved in the mixture of 3-pentanone (0.8 M) and water (1.6 M) and stirred at 100° C. for 48 h. The mixture was allowed to reach room temperature and basified with 3.0 M aqueous potassium hydroxide solution. The aqueous layer was collected, acidified with concentrated hydrochloric acid, and diluted with ethyl acetate. The organic layer was washed with brine, dried over magnesium sulfate, filtered and concentrated. No further purification was needed to afford (+/−)-3-(4-hydroxy-phenyl)-hex-4-ynoic acid.

### Step 4: (3S)-3-(4-Hydroxy-phenyl)-hex-4-ynoic Acid

A solution of (1S,2R)-1-amino-2-indanol (0.6 eq.) in acetonitrile anhydrous (0.8 M) was added to a solution of (+/−)-3-(4-hydroxy-phenyl)-hex-4-ynoic acid (1.0 eq.) in acetonitrile anhydrous (0.8 M) at 70° C. and stirred for 4 h. The mixture was allowed to reach room temperature, the salt was filtered. The salt was added in mixture of acetonitrile (0.4 M) and water (4.3 M) at 70° C. and stirred for 4 h. The reaction mixture was allowed to reach room temperature, and the salt was filtered. After two runs in the same manner, the salt was added in the mixture of ethyl acetate and water at room temperature. 2.0 M aqueous hydrochloric acid solution was added, and the mixture was stirred vigorously at room temperature. After two clear layers were obtained, the layers were separated and diluted with ethyl acetate. The organic layer was washed with brine, dried over magnesium sulfate, filtered and concentrated. No further purification was needed to afford (3S)-3-(4-hydroxy-phenyl)-hex-4-ynoic acid.

### Step 5: (3S)-3-(4-Hydroxy-phenyl)-hex-4-ynoic Acid methyl ester

Concentrated sulfuric acid (5 drops) was added to the mixture of (3 S)-3-(4-hydroxy-phenyl)-hex-4-ynoic acid (1.0 eq.) in methanol (0.5 M) at room temperature. The mixture was stirred at 90° C. for 18 h. The mixture was allowed to reach room temperature and basified with saturated aqueous sodium bicarbonate solution and extracted with ethyl acetate. The organic layer was washed with water and brine, dried over magnesium sulfate, filtered and concentrated. No further purification was needed to afford (3S)-3-(4-hydroxy-phenyl)-hex-4-ynoic acid methyl ester.

### <Intermediate 2> (S)-((4-Bromo-7-fluoro-2,3-dihydro-1H-inden-1-yl)oxy)(tert-butyl)dimethylsilane

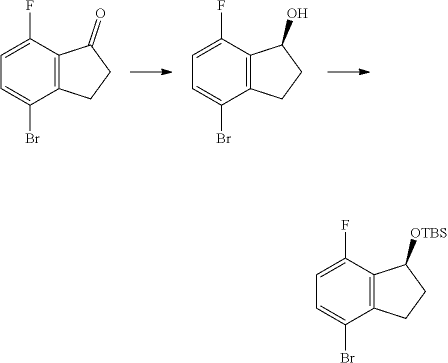 [SMILES: O=C1CCc2c(Br)ccc(F)c21 ; O[C@H]1CCc2c(Br)ccc(F)c21 ; CC(C)(C)[Si](C)(C)O[C@H]1CCc2c(Br)ccc(F)c21]

### Step 1: (S)-4-Bromo-7-fluoro-2,3-dihydro-1H-inden-1-ol

Formic acid (3.5 eg.) was added to a solution of triethylamine (3.0 eg.) in dichloromethane (1.5 M) at 4° C. 4-Bromo-7-fluoro-2,3-dihydro-1H-inden-1-one (1.0 eq.) was added and then purged with N₂ for 5 min. Chloro{[(1S,2S)-(−)-2-amino-1,2-diphenylethyl](4-toluenesulfonyl)amido}-(mesitylene)ruthenium(II) (0.02 eq.) was added and then stirred at room temperature for 18 h. The mixture was diluted with dichloromethane and washed with water. The organic layer was washed with brine, dried over magnesium sulfate, filtered and concentrated. The resultant residue was purified by flash column chromatography on silica gel to afford (S)-4-bromo-7-fluoro-2,3-dihydro-1H-inden-1-ol. Enantiomeric excess was confirmed by <Chiral UPCC analysis method I>.

### Step 2: (S)-((4-Bromo-7-fluoro-2,3-dihydro-1H-inden-1-yl)oxy)(tert-butyl)dimethylsilane

Imidazole (3.0 eq.) was added to a solution of (S)-4-bromo-7-fluoro-2,3-dihydro-1H-inden-1-ol (1.0 eq.) in dichloromethane (1.5 M) at 4° C. The reaction mixture was stirred at room temperature for 15 min. tert-Butyldimethylsilyl chloride (2.0 eq.) was added and then the mixture was allowed to reach room temperature, stirred for 1 h. The mixture was diluted with ethyl acetate and washed with water. The organic layer was washed with brine and dried over magnesium sulfate, filtered and concentrated. The resultant residue was purified by flash column chromatography on silica gel to afford (S)-((4-bromo-7-fluoro-2,3-dihydro-1H-inden-1-yl)oxy)(tert-butyl)dimethylsilane.

### <Intermediate 3> (S)-4-Bromo-1-(((tert-butyldimethylsilyl)oxy)-2,3-dihydro-1H-indene-5-carbonitrile

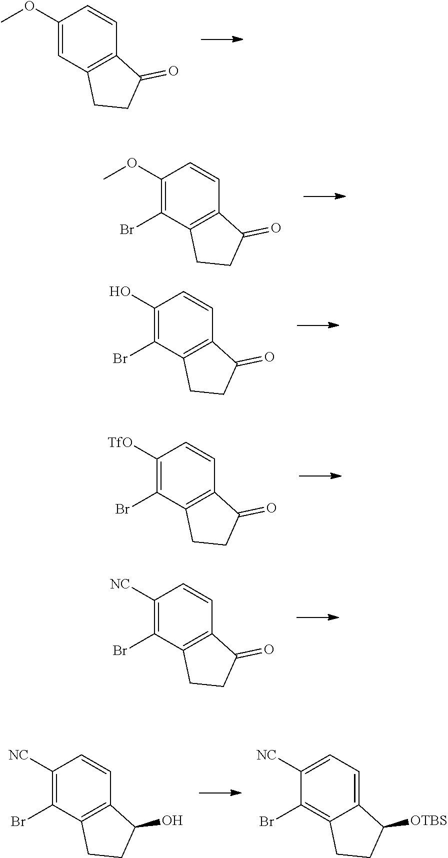

### Step 1: 4-Bromo-5-methoxy-2,3-dihydro-1H-inden-1-one

N-Bromosuccinimide (1.0 eq.) was added to a solution of 5-methoxy-2,3-dihydro-1H-inden-1-one (1.0 eq.) in water (0.1 M) and the reaction mixture was heated to 60° C. 40% aqueous sulfuric acid solution (2.0 eq.) was added and stirred at 60° C. for 6 h. The crude product was extracted with tert-butyl methyl ether and dried over magnesium sulfate, filtered and concentrated. Then, the mixture was additionally purified by crystallization using ethanol to give pure 4-bromo-5-methoxy-2,3-dihydro-1H-inden-1-one.

### Step 2: 4-Bromo-5-hydroxy-2,3-dihydro-1H-inden-1-one

Sodium thiomethoxide (4.4 eq.) was added to a solution of 4-bromo-5-methoxy-2,3-dihydro-1H-inden-1-one (1.0 eq.) in N,N-dimethylformamide (1.7 M). The reaction mixture was stirred at 120° C. for 3 h. The mixture was allowed to reach room temperature, neutralized with 1.0 M hydrochloride solution and diluted with ethyl acetate. The organic layer was washed with water and brine, dried over magnesium sulfate, filtered and concentrated. The resultant residue was purified by flash column chromatography on silica gel to afford 4-bromo-5-hydroxy-2,3-dihydro-1H-inden-1-one.

### Step 3: 4-Bromo-1-oxo-2,3-dihydro-1H-inden-5-yl trifluoromethanesulfonate

Trifluoromethanesulfonic anhydride (1.1 eq.) was added dropwise to a solution of 2,6-lutidine (2.5 eq.) and 4-bromo-5-hydroxy-2,3-dihydro-1H-inden-1-one (1.0 eq.) in dichloromethane (3.5 M) at 4° C. The reaction mixture was allowed to reach room temperature and stirred for 3 h. The mixture was diluted with dichloromethane and washed with saturated aqueous ammonium chloride solution. The organic layer was washed with water and brine, dried over magnesium sulfate, filtered and concentrated. The resultant residue was purified by flash column chromatography on silica gel to afford 4-bromo-1-oxo-2,3-dihydro-1H-inden-5-yl trifluoromethanesulfonate.

### Step 4: 4-Bromo-1-oxo-2,3-dihydro-1H-indene-5-carbonitrile

Zinc cyanide (0.3 eq.), tris(dibenzylideneacetone)dipalladium(0) (0.05 eq.) and 1,1-bis(diphenylphosphino)ferrocene (0.1 eq.) were added to a solution of 4-bromo-1-oxo-2,3-dihydro-1H-inden-5-yl trifluoromethanesulfonate (1.0 eq.) in N,N-dimethylformamide (0.6 M). The reaction mixture was stirred at 70° C. for 1 h. The mixture was diluted with ethyl acetate and washed with water. The organic layer was washed with brine, dried over magnesium sulfate, filtered and concentrated. The resultant residue was purified by flash column chromatography on silica gel to afford 4-bromo-1-oxo-2,3-dihydro-1H-indene-5-carbonitrile.

### Step 5: (S)-4-Bromo-1-hydroxy-2,3-dihydro-1H-indene-5-carbonitrile

Formic acid (3.5 eq.) was added to a solution of triethylamine (3.0 eq.) in dichloromethane (0.2 M) at 4° C. 4-Bromo-1-oxo-2,3-dihydro-1H-indene-5-carbonitrile (1.0 eq.) was added and then purged with N₂ for 5 min. Chloro{[(1S,2S)-(−)-2-amino-1,2-diphenylethyl](4-toluenesulfonyl)amido}-(mesitylene)ruthenium(II) (0.02 eq.) was added and stirred at room temperature for 18 h. The mixture was diluted with dichloromethane and washed with water. The organic layer was washed with brine, dried over magnesium sulfate, filtered and concentrated. The resultant residue was purified by flash column chromatography on silica gel to afford (S)-4-bromo-1-hydroxy-2,3-dihydro-1H-indene-5-carbonitrile. Enantiomeric excess was confirmed by <Chiral UPCC analysis method I>.

### Step 6: (S)-4-Bromo-1-((tert-butyldimethylsilyl)oxy)-2,3-dihydro-1H-indene-5-carbonitrile

Imidazole (5.0 eq.) was added to a solution of (S)-4-bromo-1-hydroxy-2,3-dihydro-1H-indene-5-carbonitrile (1.0 eq.) in dichloromethane (0.1 M) at 4° C. The reaction mixture was stirred at room temperature for 15 min. tert-Butyldimethylsilyl chloride (5.0 eq.) was added and then the reaction mixture was allowed to reach room temperature and stirred for 1 h. The mixture was diluted with ethyl acetate and washed with water. The organic layer was washed with brine and dried over magnesium sulfate, filtered and concentrated. The resultant residue was purified by flash column chromatography on silica gel to afford (S)-4-bromo-1-((tert-butyldimethylsilyl) oxy)-2,3-dihydro-1H-indene-5-carbonitrile.

### <Intermediate 4> (S)-((4-Bromo-5-fluoro-2,3-dihydro-1H-inden-1-yl)oxy)(tert-butyl)dimethylsilane

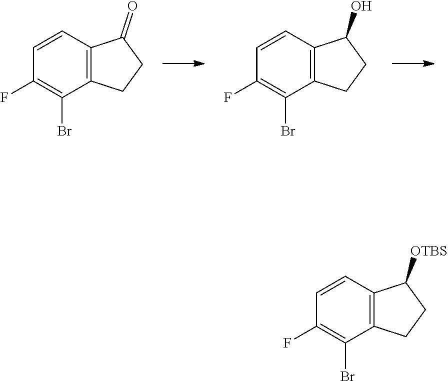 [SMILES: O=C1CCc2c1ccc(F)c2Br ; O[C@H]1CCc2c1ccc(F)c2Br ; CC(C)(C)[Si](C)(C)O[C@H]1CCc2c1ccc(F)c2Br]

### Step 1: (S)-4-Bromo-5-fluoro-2,3-dihydro-1H-inden-1-ol

Formic acid (3.5 eq.) was added to a solution of triethylamine (3.0 eq.) in dichloromethane (0.2 M) at 4° C. 4-Bromo-5-fluoro-2,3-dihydro-1H-inden-1-one (1.0 eq.) was added and then purged with N₂ for 5 min. Chloro{[(1S,2S)-(−)-2-amino-1,2-diphenylethyl](4-toluenesulfonyl)amido}-(mesitylene)ruthenium(II) (0.02 eq.) was added and stirred at room temperature for 18 h. The mixture was diluted with dichloromethane and washed with water. The organic layer was washed with brine and dried over magnesium sulfate, filtered and concentrated. The resultant residue was purified by flash column chromatography on silica gel to afford (S)-4-bromo-5-fluoro-2,3-dihydro-1H-inden-1-ol. Enantiomeric excess was confirmed by <Chiral UPCC analysis method I>.

### Step 2: (S)-((4-Bromo-5-fluoro-2,3-dihydro-1H-inden-1-yl)oxy)(tert-butyl)dimethylsilane

Imidazole (3.0 eq.) was added to a solution of (S)-4-bromo-5-fluoro-2,3-dihydro-1H-inden-1-ol (1.0 eq.) in dichloromethane (1.5 M) at 4° C. The reaction mixture was stirred at room temperature for 15 min. tert-Butyldimethylsilyl chloride (2.0 eq.) was added and then the reaction mixture was allowed to reach room temperature, stirred for 18 h. The mixture was diluted with ethyl acetate and washed with water. The organic layer was washed with brine and dried over magnesium sulfate, filtered and concentrated. The resultant residue was purified by flash column chromatography on silica gel to afford (S)-((4-bromo-5-fluoro-2,3-dihydro-1H-inden-1-yl)oxy)(tert-butyl)dimethylsilane.

### <intermediate 5> (S)-((4-Bromo-5-methoxy-2,3-dihydro-1H-inden-1-yl)oxy)(tert-butyl)dimethylsilane

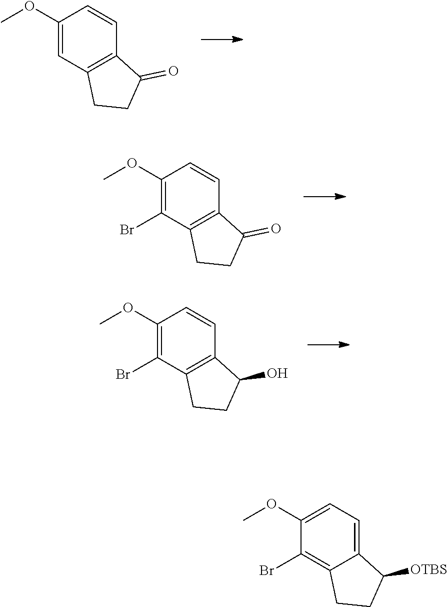

### Step 1: 4-Bromo-5-methoxy-2,3-dihydro-1H-inden-1-one

N-Bromosuccinimide (1.0 eq.) was added to a solution of 5-methoxy-2,3-dihydro-1H-inden-1-one (1.0 eq.) in water (0.1 M) and the reaction mixture was heated to 60° C. 40% aqueous sulfuric acid solution (2.0 eq.) was added and stirred for 6 h. The crude product was extracted with tert-butyl methyl ether and dried over magnesium sulfate, filtered and concentrated. Then the mixture was additionally purified by crystallization using ethanol to afford pure 4-bromo-5-methoxy-2,3-dihydro-1H-inden-1-one.

### Step 2: (S)-4-Bromo-5-methoxy-2,3-dihydro-1H-inden-1-ol

Formic acid (3.5 eq.) was added to a solution of triethylamine (3.0 eq.) in dichloromethane (0.2 M) at 4° C. 4-Bromo-5-methoxy-2,3-dihydro-1H-inden-1-one (1.0 eq.) was added and then the mixture was purged with N₂ for 5 min. Chloro{[(1S,2S)-(−)-2-amino-1,2-diphenylethyl](4-toluenesulfonyl)amido}-(mesitylene)ruthenium(II) (0.02 eq.) was added and stirred at room temperature for 18 h. The mixture was diluted with dichloromethane and washed with water. The organic layer was washed with brine and dried over magnesium sulfate, filtered and concentrated. The resultant residue was purified by flash column chromatography on silica gel to afford (S)-4-bromo-5-methoxy-2,3-dihydro-1H-inden-1-ol. Enantiomeric excess was confirmed by <Chiral UPCC analysis method I>.

### Step 3: (S)-((4-Bromo-5-methoxy-2,3-dihydro-1H-inden-1-yl)oxy)(tert-butyl)dimethylsilane

Imidazole (5.0 eq.) was added to a solution of (S)-4-bromo-5-methoxy-2,3-dihydro-1H-inden-1-ol (1.0 eq.) in dichloromethane (0.1 M) at 4° C. The reaction mixture was stirred at room temperature for 15 min. tert-Butyldimethylsilyl chloride (5.0 eq.) was added and then the reaction mixture was allowed to reach room temperature, stirred for 1 h. The mixture was diluted with ethyl acetate and washed with water. The organic layer was washed with brine and dried over magnesium sulfate, filtered and concentrated. The resultant residue was purified by flash column chromatography on silica gel to afford (S)-((4-bromo-5-methoxy-2,3-dihydro-1H-inden-1-yl)oxy)(tert-butyl)dimethylsilane.

### <intermediate 6> (S)-5-(1-tert-Butyldimethylsilyl)oxy)-5-methoxy-2,3-dihydro-1H-inden-4-yl)-2-((tetrahydro-2H-pyran-4-yl)oxy)pyridine

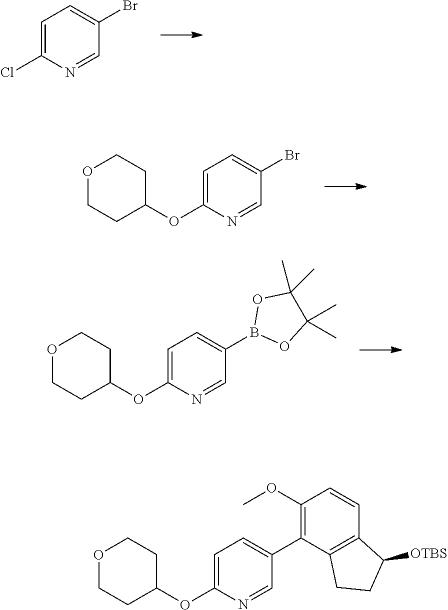 [SMILES: Clc1ccc(Br)cn1 ; Brc1ccc(OC2CCOCC2)nc1 ; CC1(C)OB(c2ccc(OC3CCOCC3)nc2)OC1(C)C ; COc1ccc2c(c1-c1ccc(OC3CCOCC3)nc1)CC[C@@H]2O[Si](C)(C)C(C)(C)C]

### Step 1: 5-Bromo-2-((tetrahydro-2H-pyran-4-yl)oxy)pyridine

Sodium hydride (1.3 eq.) was slowly added to a solution of tetrahydro-2H-pyran-4-ol (1.0 eq.) and 5-bromo-2-chloropyridine (1.2 eq.) in N,N-dimethylformamide (0.8 M) and the reaction mixture was stirred at 60° C. for 18 h. The reaction mixture was allowed to reach room temperature, quenched with water. The mixture was diluted with ethyl acetate and washed with water. The organic layer was washed with brine and dried over magnesium sulfate, filtered and concentrated. The resultant residue was purified by flash column chromatography on silica gel to afford 5-bromo-2-((tetrahydro-2H-pyran-4-yl)oxy)pyridine.

### Step 2: 2-((Tetrahydro-2H-pyran-4-yl)oxy)-5-(4,4,5,5-tetramethyl-1,3,2-dioxaborolan-2-yl)pyridine

Potassium acetate (2.0 eq.) was added to a solution of 5-bromo-2-((tetrahydro-2H-pyran-4-yl) oxy) pyridine (1.0 eq.) and bis(pinacolato)diboron (1.2 eq.) in 1,4-dioxane (0.1 M) and then purged with N₂ for 10 min. The reaction mixture was stirred at 110° C. for 18 h. The reaction mixture was allowed to reach room temperature, diluted with ethyl acetate and washed with water. The organic layer was washed with brine and dried over magnesium sulfate, filtered and concentrated. The resultant residue was purified by flash column chromatography on silica gel to afford 2-((tetrahydro-2H-pyran-4-yl)oxy)-5-(4,4,5,5-tetramethyl-1,3,2-dioxaborolan-2-yl)pyridine.

### Step 3: (S)-5-(1-((tert-Butyldimethylsilyl)oxy)-5-methoxy-2,3-dihydro-1H-inden-4-yl)-2-((tetrahydro-2H-pyran-4-yl)oxy)pyridine

Tris(dibenzylideneacetone)dipalladium(0) (0.05 eq.) was added to a solution of (S)-((4-bromo-5-methoxy-2,3-dihydro-1H-inden-1-yl)oxy)(tert-butyl)dimethylsilane (1.0 eq.), 2-((tetrahydro-2H-pyran-4-yl)oxy)-5-(4,4,5,5-tetramethyl-1,3,2-dioxaborolan-2-yl)pyridine (1.2 eq.), 2-dicyclohexylphosphino-2′,6′-dimethoxybiphenyl (0.1 eq.), and potassium phosphate tribasic (3.0 eq.) in toluene (0.1 M) and water (1.0 M) and then purged with N₂ for 10 min. The reaction mixture was stirred at 120° C. for 18 h under N₂ atmosphere. The reaction mixture was allowed to reach room temperature, filtered through Celite. The filtrate was diluted with ethyl acetate and washed with water. The organic layer was washed with brine and dried over magnesium sulfate, filtered and concentrated. The resultant residue was purified by flash column chromatography on silica gel to afford (S)-5-(1-((tert-butyldimethylsilyl)oxy)-5-methoxy-2,3-dihydro-1H-inden-4-yl)-2-((tetrahydro-2H-pyran-4-yl)oxy)pyridine.

### <Intermediate 7>3-(6-Hydroxypyridin-3-yl)hex-4-ynoic acid ethyl ester

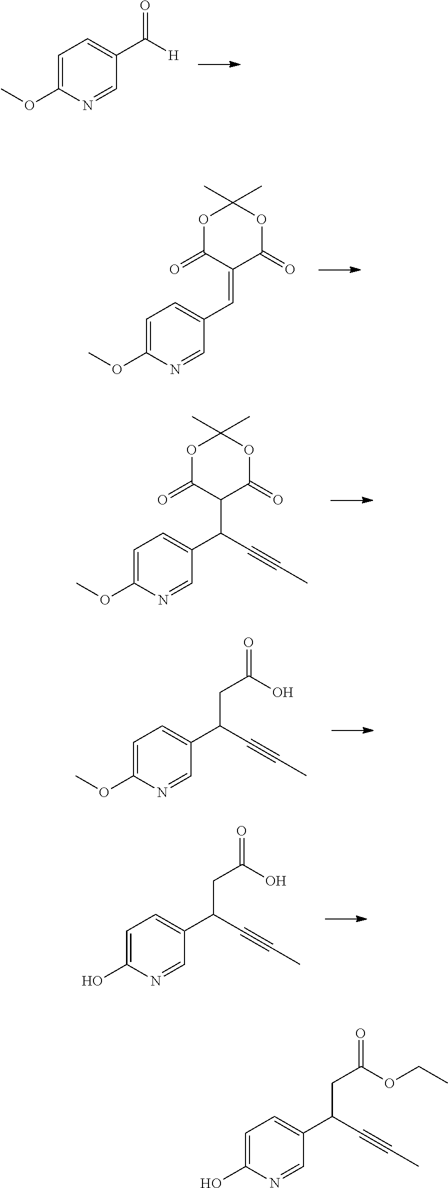

### Step 1: 5-((6-Methoxypyridin-3-yl)methylene)-2,2-dimethyl-1,3-dioxane-4,6-dione

6-Methoxypyridine-3-carbaldehyde (1.0 eq.) was dissolved in water (0.9 M) at 75° C. Subsequently, a solution of Meldrum's acid (1.1 eq.) in water (1.2 M) was added to the mixture. The mixture was stirred at 75° C. for 2 h, and then additional portion of Meldrum's acid (0.5 eq.) in water (1.2 M) was added to the reaction mixture. The reaction mixture was stirred at 75° C. for 2 h. The mixture was allowed to reach room temperature, filtered with iced water. The wet solid resultant was dried in an oven (50° C.) to afford 5-((6-methoxypyridin-3-yl) methylene)-2,2-dimethyl-1,3-dioxane-4,6-dione.

### Step 2: 5-(l-(6-Methoxypyridin-3-yl)but-2-ynyl)-2,2-dimethyl-1,3-dioxane-4,6-dione

A solution of 5-((6-methoxypyridin-3-yl)methylene)-2,2-dimethyl-1,3-dioxane-4,6-dione (1.0 eq.) in tetrahydrofuran anhydrous (0.4 M) was added dropwise to 1-propynylmagnesium bromide in tetrahydrofuran (0.5 N, 1.5 eq.) at 4° C. under N₂ atmosphere. The mixture was stirred at room temperature for 30 min. The mixture was quenched with saturated aqueous ammonium chloride solution and extracted with hexane. After the aqueous layer was collected, the mixture was acidified with 1.0 M aqueous hydrochloric acid solution and diluted with ethyl acetate. The organic layer was washed with water and brine, dried over magnesium sulfate, filtered and concentrated. The resultant residue was purified by flash column chromatography on silica gel eluting to afford 5-(l-(6-methoxypyridin-3-yl)but-2-ynyl)-2,2-dimethyl-1,3-dioxane-4,6-dione.

### Step 3: 3-(6-Methoxypyridin-3-yl)hex-4-ynoic Acid

5-(l-(6-Methoxypyridin-3-yl)but-2-ynyl)-2,2-dimethyl-1,3-dioxane-4,6-dione (1.0 eq.) was dissolved in the mixture of N,N-dimethylformamide (0.2 M) and water (2.0 M) at 100° C. and stirred for 18 h. The reaction mixture was allowed to reach room temperature, quenched with saturated aqueous ammonium chloride solution and the extracted with ethyl acetate. The organic layer was washed with brine, dried over magnesium sulfate, filtered and concentrated. No further purification was needed to afford 3-(6-methoxypyridin-3-yl)hex-4-ynoic acid.

### Step 4: 3-(6-Hydroxypyridin-3-yl) hex-4-ynoic Acid

Concentrated hydrochloric acid solution (8.0 M) was added to the mixture of 3-(6-methoxypyridin-3-yl)hex-4-ynoic acid (1.0 eq.) in 1,4-dioxane (2.0 M) and water (2.0 M) at room temperature. The mixture was stirred at 100° C. for 18 h under N₂ atmosphere. The reaction mixture was allowed to reach room temperature, basified with saturated aqueous sodium bicarbonate solution and extracted with ethyl acetate. The organic layer was washed with brine, dried over magnesium sulfate, filtered and concentrated. No further purification was needed to afford 3-(6-hydroxypyridin-3-yl) hex-4-ynoic acid.

### Step 5: 3-(6-Hydroxypyridin-3-yl) hex-4-ynoic Acid ethyl ester

Concentrated sulfuric acid (5 drops) was added to the mixture of 3-(6-hydroxypyridin-3-yl) hex-4-ynoic acid (1.0 eq.) in ethanol (0.9 M) at room temperature. The mixture was stirred at 90° C. for 18 h. The reaction mixture was allowed to reach room temperature, basified with saturated aqueous sodium bicarbonate solution and extracted with ethyl acetate. The organic layer was washed with water and brine, dried over magnesium sulfate, filtered and concentrated. No further purification was needed to afford 3-(6-hydroxypyridin-3-yl) hex-4-ynoic acid ethyl ester.

### <Intermediate 8> (S)-3-(4-(((R)-4-(5-Chloro-6-((tetrahydro-2H-pyran-4-yl)oxy)pyridin-3-yl)-7-fluoro-2,3-dihydro-1H-inden-1-yl)oxy)phenyl)hex-4-ynoic Acid methyl ester

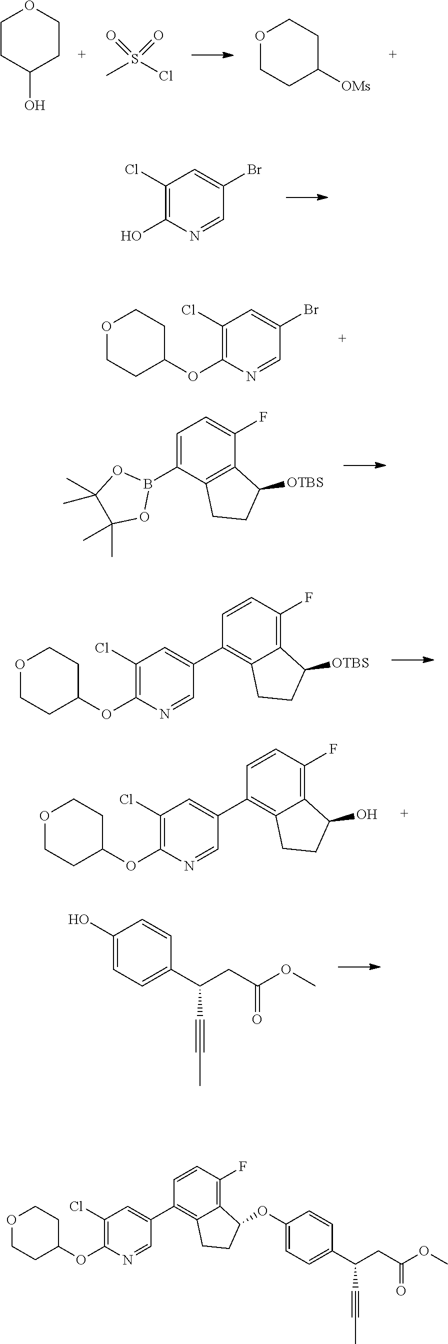

### Step 1: Tetrahydro-2H-pyran-4-yl methanesulfonate

Methanesulfonyl chloride (1.2 eq.) was added to a solution of tetrahydro-2H-pyran-4-ol (1.0 eq.) and triethylamine (3.0 eq.) in dichloromethane (0.3 M) at 4° C. The reaction mixture was stirred at room temperature for 1 h. The mixture was diluted with dichloromethane and washed with water. The organic layer was washed with brine, saturated aqueous ammonium chloride, and dried over magnesium sulfate, filtered and concentrated. The resultant residue was purified by flash column chromatography on silica gel to afford tetrahydro-2H-pyran-4-yl methanesulfonate.

### Step 2: 5-Bromo-3-chloro-2-((tetrahydro-2H-pyran-4-yl)oxy)pyridine

Potassium carbonate (2.0 eq.) was added to a solution of tetrahydro-2H-pyran-4-yl methanesulfonate (1.2 eq.) and 5-bromo-3-chloropyridine-1-ol (1.0 eq.) in N,N-dimethylformamide (0.2 M). The reaction mixture was stirred at 100° C. for 18 h. The reaction mixture was allowed to reach room temperature, diluted with ethyl acetate and washed with water. The organic layer was washed with brine, dried over magnesium sulfate, filtered and concentrated. The resultant residue was purified by flash column chromatography on silica gel to afford 5-bromo-3-chloro-2-((tetrahydro-2H-pyran-4-yl) oxy) pyridine.

### Step 3: (S)-5-(1-((tert-Butyldimethylsilyl)oxy)-7-fluoro-2,3-dihydro-1H-inden-4-yl)-3-chloro-2-((tetrahydro-2H-pyran-4-yl)oxy)pyridine

Tris(dibenzylideneacetone)dipalladium(0) (0.05 eq.) was added to a solution of 5-bromo-3-chloro-2-((tetrahydro-2H-pyran-4-yl)oxy)pyridine (1.0 eq.), (S)-tert-butyl((7-fluoro-4-(4,4,5,5-tetramethyl-1,3,2-dioxaborolan-2-yl)-2,3-dihydro-1H-inden-1-yl)oxy)dimethylsilane (1.2 eq.), 2-dicyclohexylphosphino-2′,6′-dimethoxybiphenyl (0.1 eq.), and potassium phosphate tribasic (3.0 eq.) in toluene (0.1 M) and water (1.0 M) and then purged with N₂ atmosphere for 10 min. The reaction mixture was stirred at 120° C. for 18 h under N₂ atmosphere. The reaction mixture was allowed to reach room temperature, filtered through Celite. The filtrate was diluted with ethyl acetate and washed with water. The organic layer was washed with brine, dried over magnesium sulfate, filtered and concentrated. The resultant residue was purified by flash column chromatography on silica gel to afford (S)-5-(1-((tert-butyldimethylsilyl)oxy)-7-fluoro-2,3-dihydro-1H-inden-4-yl)-3-chloro-2-((tetrahydro-2H-pyran-4-yl)oxy)pyridine.

### Step 4: (S)-4-(5-Chloro-6-((tetrahydro-2H-pyran-4-yl)oxy)pyridin-3-yl)-7-fluoro-2,3-dihydro-1H-inden-1-ol

1.0 M Tetra-n-butyl ammonium fluoride (2.0 eq.) was added dropwise to a solution of (S)-5-(1-((tert-butyldimethylsilyl)oxy)-7-fluoro-2,3-dihydro-1H-inden-4-yl)-3-chloro-2-((tetrahydro-2H-pyran-4-yl)oxy)pyridine (1.0 eq.) in tetrahydrofuran (0.1 M) at 4° C. The reaction mixture was stirred at room temperature for 4 h. The mixture was diluted with ethyl acetate and washed with water. The organic layer was washed with brine and dried over magnesium sulfate, filtered and concentrated. The resultant residue was purified by flash column chromatography on silica gel to afford (S)-4-(5-chloro-6-((tetrahydro-2H-pyran-4-yl)oxy)pyridin-3-yl)-7-fluoro-2,3-dihydro-1H-inden-1-ol.

### Step 5: (S)-3-(4-(((R)-4-(5-Chloro-6-((tetrahydro-2H-pyran-4-yl)oxy)pyridin-3-yl)-7-fluoro-2,3-dihydro-1H-inden-1-yl)oxy)phenyl)hex-4-ynoic acid methyl ester

1,1′-(Azodicarbonyl)dipiperidine (1.5 eq.) was added portion wise over 10 min to a solution of (S)-4-(5-chloro-6-((tetrahydro-2H-pyran-4-yl)oxy)pyridin-3-yl)-7-fluoro-2,3-dihydro-1H-inden-1-ol (1.0 eq.), (3S)-3-(4-hydroxy-phenyl)-hex-4-ynoic acid methyl ester (1.0 eq.), and tri-n-butylphosphine (1.5 eq.) in toluene (0.1 M) at 4° C. The mixture was stirred at room temperature for 18 h. After addition of hexane (0.05 M) to reaction mixture, the resulting white solid was removed by filtration. The filtrate was concentrated and then purified by flash column chromatography on silica gel to afford (S)-3-(4-(((R)-4-(5-chloro-6-((tetrahydro-2H-pyran-4-yl)oxy)pyridin-3-yl)-7-fluoro-2,3-dihydro-1H-inden-1-yl)oxy)phenyl)hex-4-ynoic acid methyl ester.

### <Intermediate 9> Chiral UPCC Analysis Method I

Flow rate: 2 mL/min.
Mobile phase: Isocratic CO₂/Ethanol (80/20)
Stationary phase: CHIRALPAK-IA 250*4.6 mm I.D.

### Temperature: 25° C.

### Absorbance Wavelength: 220 nm

### <Intermediate 10> (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic Acid methyl ester

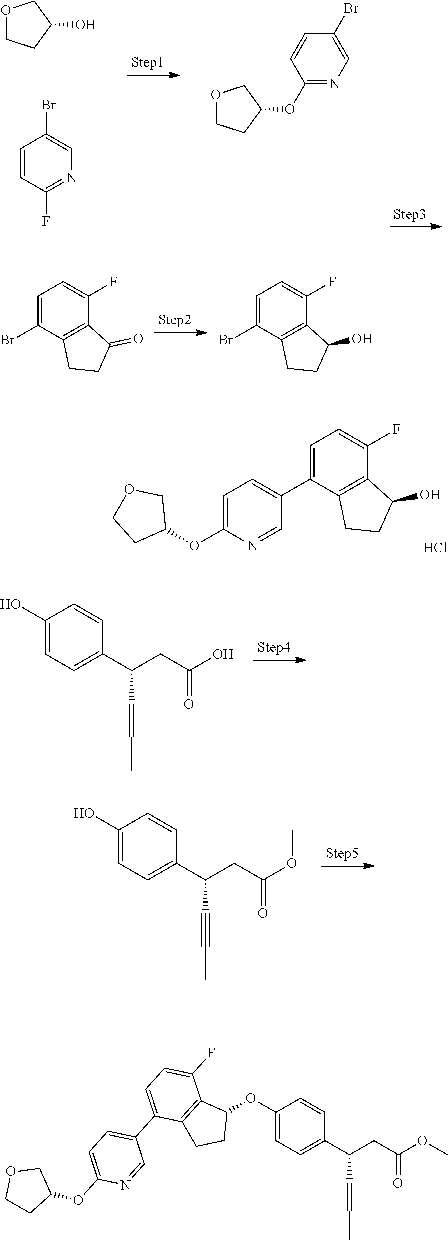

### Step 1: (R)-5-bromo-2-((tetrahydrofuran-3-yl)oxy)pyridine

Potassium hydroxide (1.5 eq.) was added to a solution of (R)-tetrahydrofuran-3-ol (1.05 eq.) and -bromo-2-fluoropyridine (1.0 eq.) in acetonitrile at room temperature. The reaction mixture was refluxed for 1 hour. The reaction mixture was evaporated at reduced pressure to remove solution. The mixture was diluted with ethyl acetate and washed with water. The organic layer was washed with 10% NaCl solution, and dried over magnesium sulfate, filtered and concentrated. Water was added dropwise to the resultant residue to obtain (R)-5-bromo-2-((tetrahydrofuran-3-yl)oxy)pyridine as white to almost white crystalline powder.

### Step 2: (S)-4-bromo-7-fluoro-2,3-dihydro-1H-inden-1-ol

Formic acid (3.5 eq.) was slowly added to a solution of -bromo-7-fluoro-2,3-dihydro-1H-inden-1-one (1.0 eq.), triethylamine (3.0 eq.), and [(S,S)—N-(2-amino-1,2-diphenylethyl)-p-toluenesulfonamide]chloro(mesitylene)ruthenium(II) (0.001 eq.) in dichloromethane at −10° C. The reaction mixture was refluxed for 2 hours at 65° C. The reaction mixture was evaporated at reduced pressure to remove solution. The mixture was diluted with ethyl acetate and washed with water. The organic layer was washed with 1N hydrochloride solution and 1N sodium bicarbonate solution. The organic layer was dried over magnesium sulfate and active C, filtered and concentrated. n-Hexane was added to the resultant residue to obtain (S)-4-bromo-7-fluoro-2,3-dihydro-1H-inden-1-ol as white to light gray crystals.

### Step 3: (S)-7-fluoro-4-(6-(((R)-tetrahydrofuran-3-yl)oxy)pyridin-3-yl)-2,3-dihydro-1H-inden-1-ol hydrogen chloride

1,1′-bis(diphenylphosphino)ferrocene-palladium(II) dichloride dichloromethane complex (0.05 eq.) was added to a solution of (R)-5-bromo-2-((tetrahydrofuran-3-yl)oxy)pyridine (1.0 eq.), bis(pinacolato)diboron (1.1 eq.), and potassium acetate (2.0 eq.) in 1,4-dioxane at room temperature. The reaction mixture was refluxed for 2 hours. (S)-4-bromo-7-fluoro-2,3-dihydro-1H-inden-1-ol (1.0 eq.) was added and was refluxed for 2 hours. The reaction mixture was evaporated at reduced pressure to remove solution. The mixture was diluted with ethyl acetate and washed with water. The organic layer was washed with 10% NaCl solution. The organic layer was dried over magnesium sulfate and active C, filtered and concentrated. Methanol and acetone were added to the resultant residue to obtain (S)-7-fluoro-4-(6-(((R)-tetrahydrofuran-3-yl)oxy)pyridin-3-yl)-2,3-dihydro-1H-inden-1-ol hydrogen chloride as white to almost white crystalline powder.

### Step 4: (S)-3-(4-hydroxyphenyl)hex-4-ynoic acid methyl ester

Sulfuric acid (0.1 eq.) was added to a solution of (S)-3-(4-hydroxyphenyl)hex-4-ynoic acid (1.0 eq.) in methanol at room temperature. The reaction mixture was refluxed for 4 hours. The reaction mixture was evaporated at reduced pressure to remove solution. The mixture was diluted with ethyl acetate and washed with water. The organic layer was washed with 10% NaCl solution. The organic layer was dried over magnesium sulfate, filtered and concentrated. Toluene was added to the residue and re-concentrated to obtain (S)-3-(4-hydroxyphenyl)hex-4-ynoic acid methyl ester in oil form.

### Step 5: (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid methyl ester

1N Sodium hydroxide solution was added a solution of (S)-7-fluoro-4-(6-(((R)-tetrahydrofuran-3-yl)oxy)pyridin-3-yl)-2,3-dihydro-1H-inden-1-ol hydrogen chloride (1.0 eq.) in dichloromethane and water. The organic layer was dried over magnesium sulfate, filtered and concentrated to obtain (S)-7-fluoro-4-(6-(((R)-tetrahydrofuran-3-yl)oxy)pyridin-3-yl)-2,3-dihydro-1H-inden-1-ol.

(S)-3-(4-hydroxyphenyl)hex-4-ynoic acid methyl ester in toluene, tri-n-butyl phosphine (1.3 eq.) and 1,1′-(azodicarbonyl)dipiperidine. (1.3 eq.) were added a solution of (S)-7-fluoro-4-(6-(((R)-tetrahydrofuran-3-yl)oxy)pyridin-3-yl)-2,3-dihydro-1H-inden-1-ol. The reaction mixture is stirred at room temperature for one day. n-Hexane is added to the resulting mixture and filtered out by-product. Concentrate the filtrate and n-hexane was added to the residue to obtain (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid methyl ester as off-white crystal powder.

### EXAMPLES

### <Example 1> (S)-3-(4-(((R)-4-(6-((1,1-Dioxidotetrahydro-2H-thiopyran-4-yl)oxy)pyridin-3-yl)-7-fluoro-2,3-dihydro-1H-inden-1-yl)oxy)phenyl)hex-4-ynoic Acid

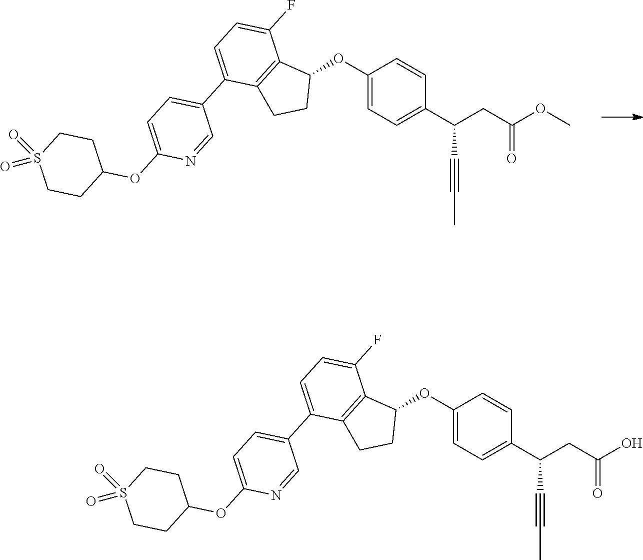 [SMILES: CC#C[C@@H](CC(=O)OC)c1ccc(O[C@@H]2CCc3c(-c4ccc(OC5CCS(=O)(=O)CC5)nc4)ccc(F)c32)cc1 ; CC#C[C@@H](CC(=O)O)c1ccc(O[C@@H]2CCc3c(-c4ccc(OC5CCS(=O)(=O)CC5)nc4)ccc(F)c32)cc1]

2.0 M aqueous lithium hydroxide solution (5.0 eq.) was added to a_solution of (S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid methyl ester (1.0 eq.) in tetrahydrofuran (1.0 M) and methanol (4.0 M) at 4° C. The mixture was stirred at room temperature for 18 h. The mixture was neutralized with saturated aqueous ammonium chloride solution and diluted with ethyl acetate. The organic layer was washed with brine, dried over magnesium sulfate, filtered, and concentrated. The resultant residue was purified by flash column chromatography on silica gel to afford (S)-3-(4-(((R)-4-(6-((1,1-dioxidotetrahydro-2H-thiopyran-4-yl)oxy)pyridin-3-yl)-7-fluoro-2,3-dihydro-1H-inden-1-yl)oxy)phenyl)hex-4-ynoic acid. MS ESI (positive) m/z: 564.15 (M+H).

¹H NMR (400 MHz, CDCl₃) δ 8.18 (s, 1H), 7.69 (d, J=8.4 Hz, 1H), 7.34 (d, J=8.4 Hz, 2H), 7.29-7.25 (m, 1H), 7.04 (t, J=8.4 Hz, 1H), 6.96 (d, J=8.4 Hz, 2H), 6.83 (d, J=8.4 Hz, 1H), 5.93 (s, 1H), 5.45 (s, 1H), 4.16-4.07 (m, 1H), 3.44-3.37 (m, 2H), 3.32-3.23 (m, 1H), 3.03-2.98 (m, 2H), 2.90-2.80 (m, 2H), 2.76-2.71 (m, 1H), 2.57-2.54 (m, 2H), 2.48-2.32 (m, 4H), 1.84 (d, J=2.4 Hz, 3H).

### <Example 2> (S)-3-(4-(((R)-7-Fluoro-4-(6-((3-methyloxetan-3-yl)methoxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(6-((3-methyloxetan-3-yl)methoxy)pyridin-3-yl)-2,3-dihydro-1H-inden-1-yl)oxy)phenyl)hex-4-ynoic acid methyl ester through the same procedure as used in Example 1. MS ESI (positive) m/z: 516.15 (M+H).

¹H NMR (400 MHz, CDCl₃) δ 8.21 (d, J=2.4 Hz, 1H), 7.67-7.65 (m, 1H), 7.33-7.28 (m, 3H), 7.03 (t, J=8.6 Hz, 1H), 6.97-6.95 (m, 2H), 6.86 (d, J=8.4 Hz, 1H), 5.93-5.91 (m, 1H), 4.68 (d, J=5.6 Hz, 2H), 4.47 (d, J=5.6 Hz, 2H), 4.42 (s, 2H), 4.09-4.08 (m, 1H), 3.28-3.26 (m, 1H), 2.92-2.85 (m, 1H), 2.81-2.65 (m, 2H), 2.39-2.36 (m, 2H), 1.84 (d, J=2.4 Hz, 3H).

### <Example 3> (S)-3-(4-(((R)-4-(6-(2-(1,1-Dioxidothiomorpholino)ethoxy)pyridin-3-yl)-7-fluoro-2,3-dihydro-1H-inden-1-yl)oxy)phenyl)hex-4-ynoic acid

The title compound was synthesized from (S)-3-(4-(((R)-4-(6-(2-(1,1-dioxidothiomorpholino)ethoxy)pyridin-3-yl)-7-fluoro-2,3-dihydro-1H-inden-1-yl)oxy)phenyl)hex-4-ynoic acid methyl ester through the same procedure as used in Example 1. MS ESI (positive) m/z: 593.08 (M+H).

¹H NMR (400 MHz, CDCl₃) δ 8.16 (d, J=2.4 Hz, 1H), 7.65 (dd, J=8.6, 2.4 Hz, 1H), 7.35-7.26 (m, 3H), 7.08-7.00 (m, 1H), 6.95 (d, J=8.4 Hz, 2H), 6.81 (d, J=8.8 Hz, 1H), 5.92 (t, J=2.6 Hz, 1H), 4.49 (t, J=5.4 Hz, 2H), 4.11-4.02 (m, 1H), 3.29-3.15 (m, 5H), 3.15-2.99 (m, 6H), 2.90-2.70 (m, 3H), 2.42-2.35 (m, 2H), 1.84 (d, J=2.4 Hz, 3H).

### <Example 4> (S)-3-(4-(((R)-7-Fluoro-4-(6-(oxetan-3-yloxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(6-(oxetan-3-yloxy)pyridin-3-yl)-2,3-dihydro-1H-inden-1-yl)oxy)phenyl)hex-4-ynoic acid methyl ester through the same procedure as used in Example 1. MS ESI (positive) m/z: 488.12 (M+H).

¹H NMR (400 MHz, CDCl₃) δ 8.12 (s, 1H), 7.67 (d, J=4.4 Hz, 1H), 7.33-7.24 (m, 3H), 7.01 (t, J=2.4 Hz, 1H), 6.98 (d, J=8.4 Hz, 2H), 6.86 (d, J=4.8 Hz, 1H), 5.92 (t, J=2.6 Hz, 1H), 5.67 (t, J=5.4 Hz, 1H), 5.04-5.01 (m, 2H), 4.79-4.76 (m, 2H), 4.09-4.06 (m, 1H), 3.29-3.25 (m, 1H), 2.81-2.79 (m, 1H), 2.78-2.75 (m, 1H), 2.74-2.70 (m, 1H), 2.39-2.35 (m, 2H), 1.84 (s, 3H).

### <Example 5> (S)-3-(4-(((R)-7-Fluoro-4-(6-(((R)-tetrahydrofuran-3-yl)oxy)pyridin-3-yl)-2,3-dihydro-1H-inden-1-yl)oxy)phenyl)hex-4-ynoic Acid

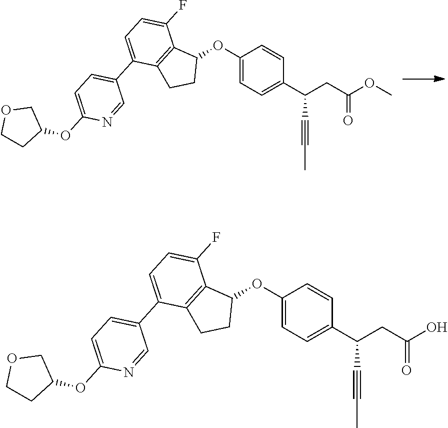

2N Lithium hydroxide solution was added a solution of (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid methyl ester in methanol and water at 10° C. The mixture was diluted with 1N hydrochloric acid solution and ethyl acetate. The organic layer was washed with NaCl solution. The organic layer was dried over magnesium sulfate, filtered and concentrated.

Acetone and n-heptane were added to the resultant residue to obtain (S)-3-(4-(((R)-7-fluoro-4-(6-(((R)-tetrahydrofuran-3-yl)oxy)pyridin-3-yl)-2,3-dihydro-1H-inden-1-yl)oxy)phenyl)hex-4-ynoic acid MS ESI (positive) m/z: 502.24 (M+H).

¹H NMR (400 MHz, CDCl₃) δ 8.15 (d, J=2.4 Hz, 1H), 7.64 (dd, J=8.6, 2.6 Hz, 1H), 7.38-7.26 (m, 3H), 7.03 (t, J=8.6 Hz, 1H), 6.98-6.93 (m, 2H), 6.81 (dd, J=8.4, 0.4 Hz, 1H), 5.95-5.91 (m, 1H), 5.61-5.58 (m, 1H), 4.11-3.89 (m, 5H), 3.29-3.19 (m, 1H), 2.91-2.71 (m, 3H), 2.42-2.15 (m, 4H), 1.84 (d, J=2.4 Hz, 3H).

### <Example 6> (S)-3-(4-(((R)-7-Fluoro-4-(6-((tetrahydro-2H-pyran-4-yl)oxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(6-((tetrahydro-2H-pyran-4-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 516.06 (M+H).

¹H NMR (400 MHz, CDCl₃) δ 8.15 (d, J=2.4 Hz, 1H), 7.63 (dd, J=8.6, 2.6 Hz, 1H), 7.33 (d, J=8.4 Hz, 2H), 7.29-7.26 (m, 1H), 7.02 (t, J=8.6 Hz, 1H), 6.95 (d, J=8.4 Hz, 2H), 6.79 (d, J=8.4 Hz, 1H), 5.92-5.90 (m, 1H), 5.26-5.25 (m, 1H), 4.07-4.02 (m, 1H), 4.00-3.98 (m, 2H), 3.65-3.60 (m, 2H), 3.25-3.21 (m, 1H), 2.86-2.80 (m, 2H), 2.75-2.70 (m, 1H), 2.37-2.34 (m, 2H), 2.11-2.07 (m, 2H), 1.85-1.80 (m, 5H).

### <Example 7> (S)-3-(4-(((R)-7-Fluoro-4-(6-(((S)-tetrahydrofuran-3-yl)oxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(6-(((S)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 502.15 (M+H).

¹H NMR (400 MHz, CDCl₃) δ 8.15 (d, J=2.4 Hz, 1H), 7.64 (dd, J=8.6, 2.6 Hz, 1H), 7.38-7.26 (m, 3H), 7.03 (t, J=8.6 Hz, 1H), 6.98-6.93 (m, 2H), 6.81 (dd, J=8.4, 0.4 Hz, 1H), 5.95-5.91 (m, 1H), 5.61-5.58 (m, 1H), 4.11-3.89 (m, 5H), 3.29-3.19 (m, 1H), 2.91-2.71 (m, 3H), 2.42-2.15 (m, 4H), 1.84 (d, J=2.4 Hz, 3H).

### <Example 8> (S)-3-(4-(((R)-7-Fluoro-4-(4-methyl-6-(((R)-tetrahydrofuran-3-yl)oxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(4-methyl-6-(((R)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 516.19 (M+H).

¹H NMR (400 MHz, CDCl₃) δ 7.78 (s, 1H), 7.31 (d, J=8.4 Hz, 2H), 7.16-7.12 (m, 1H), 7.01 (t, J=8.4 Hz, 1H), 6.91 (d, J=8.4 Hz, 2H), 6.67 (s, 1H), 5.95 (d, J=5.6 Hz, 1H), 5.58-5.52 (m, 1H), 4.10-3.84 (m, 5H), 2.88-2.71 (m, 3H), 2.65-2.47 (m, 1H), 2.44-2.22 (m, 3H), 2.20-2.11 (m, 1H), 2.11 (s, 3H), 1.84 (d, J=2.4 Hz, 3H).

### <Example 9> (S)-3-(4-(((R)-7-Fluoro-4-(2-methyl-6-(((R)-tetrahydrofuran-3-yl)oxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(2-methyl-6-(((R)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 516.11 (M+H).

¹H NMR (400 MHz, CDCl₃) δ 7.38-7.31 (m, 3H), 7.16-7.10 (m, 1H), 7.02-6.95 (m, 3H), 6.60 (d, J=8.4 Hz, 1H), 5.94 (d, J=4.8 Hz, 1H), 5.63-5.58 (m, 1H), 4.16-3.87 (m, 5H), 3.02-2.92 (m, 1H), 2.88-2.69 (m, 2H), 2.65-2.55 (m, 1H), 2.48-2.37 (m, 1H), 2.35-2.13 (m, 6H), 1.84 (d, J=2.4 Hz, 3H).

### <Example 10> (S)-3-(4-(((R)-4-(5-Chloro-6-(((R)-tetrahydrofuran-3-yl)oxy)pyridin-3-yl)-7-fluoro-2,3-dihydro-1H-inden-1-yl)oxy)phenyl)hex-4-ynoic Acid

The title compound was synthesized from (S)-3-(4-(((R)-4-(5-chloro-6-(((R)-tetrahydrofuran-3-yl)oxy)pyridin-3-yl)-7-fluoro-2,3-dihydro-1H-inden-1-yl)oxy)phenyl)hex-4-ynoic acid methyl ester through the same procedure as used in Example 1. MS ESI (positive) m/z: 536.15 (M+H).

¹H NMR (400 MHz, CDCl₃) δ 7.97 (s, 1H), 7.61 (s, 1H), 7.25 (d, J=8.4 Hz, 2H), 7.21 (t, J=8.4 Hz, 1H), 6.93 (t, J=8.4 Hz, 1H), 6.83 (t, J=8.4 Hz, 2H), 5.79 (t, J=4.6 Hz, 1H), 5.54-5.52 (m, 1H), 4.04-3.97 (m, 5H), 3.21-3.16 (m, 1H), 2.88-2.67 (m, 3H), 2.28-2.17 (m, 4H), 1.74 (s, 3H).

### <Example 11> (S)-3-(4-(((R)-7-Fluoro-4-(5-(((R)-tetrahydrofuran-3-yl)oxy)pyridin-2-yl)-2,3-dihydro-1H-inden-1-yl)oxy)phenyl)hex-4-ynoic Acid

The title compound was synthesized from (S)-3-(4-(((R)-7-fluoro-4-(5-(((R)-tetrahydrofuran-3-yl)oxy)pyridin-2-yl)-2,3-dihydro-1H-inden-1-yl)oxy)phenyl)hex-4-ynoic acid methyl ester through the same procedure as used in Example 1. MS ESI (positive) m/z: 502.20 (M+H).

¹H NMR (400 MHz, CDCl₃) δ 8.36 (s, 1H), 7.66 (dd, J=8.6, 2.6 Hz, 1H), 7.46 (d, J=4.0 Hz, 1H), 7.31 (d, J=2.0 Hz, 2H), 7.25-7.23 (m, 1H), 7.03 (t, J=8.6 Hz, 1H), 6.96 (t, J=2.6 Hz, 2H), 5.92-5.90 (m, 1H), 5.01-4.99 (m, 1H), 4.06-3.93 (m, 5H), 3.61-3.57 (m, 1H), 3.38-3.19 (m, 1H), 2.80-2.77 (m, 1H), 2.76-2.74 (m, 1H), 2.65-2.39 (m, 4H), 1.54 (s, 3H).

### <Example 12> (S)-3-(4-(((R)-7-Fluoro-4-(4-methyl-6-(Y3-methyloxetan-3-yl)methoxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(4-methyl-6-((3-methyloxetan-3-yl)methoxy)pyridin-3-yl)-2,3-dihydro-1H-inden-1-yl)oxy)phenyl)hex-4-ynoic acid methyl ester through the same procedure as used in Example 1. MS ESI (positive) m/z: 530.17 (M+H).

¹H NMR (400 MHz, CDCl₃) δ 7.91 (s, 1H), 7.32-7.30 (m, 2H), 7.15-7.12 (m, 1H), 7.02-7.00 (m, 1H), 6.98-6.94 (m, 2H), 6.77 (s, 1H), 5.95-5.93 (m, 1H), 4.68 (d, J=5.6 Hz, 2H), 4.46 (d, J=5.6 Hz, 2H), 4.38 (s, 2H), 4.09-4.08 (m, 1H), 3.03-2.90 (m, 1H), 2.78-2.65 (m, 3H), 2.43-2.37 (m, 1H), 2.32-2.27 (m, 1H), 2.10 (s, 3H), 1.83 (d, J=2.4 Hz, 3H).

### <Example 13> (S)-3-(4-(((R)-7-Fluoro-4-(2-methyl-6-(Y3-methyloxetan-3-yl)methoxy)pyridin-3-yl)-2,3-dihydro-1H-inden-1-yl)oxy)phenyl)hex-4-ynoic acid

The title compound was synthesized from (S)-3-(4-(((R)-7-fluoro-4-(2-methyl-6-((3-methyloxetan-3-yl)methoxy)pyridin-3-yl)-2,3-dihydro-1H-inden-1-yl)oxy)phenyl)hex-4-ynoic acid methyl ester through the same procedure as used in Example 1. MS ESI (positive) m/z: 530.16 (M+H).

¹H NMR (400 MHz, CDCl₃) δ 7.38-7.29 (m, 3H), 7.16-7.08 (m, 1H), 7.00-6.90 (m, 3H), 6.64 (d, J=8.4 Hz, 1H), 5.91 (d, J=5.2 Hz, 1H), 4.69 (d, J=5.8 Hz, 2H), 4.46 (d, J=5.8 Hz, 2H), 4.40 (s, 2H), 4.12-4.05 (m, 1H), 2.99-2.88 (m, 1H), 2.85-2.64 (m, 2H), 2.62-2.54 (m, 1H), 2.48-2.31 (m, 1H), 2.29-2.20 (m, 4H), 1.81 (d, J=2.4 Hz, 3H).

### <Example 14> (S)-3-(4-(((R)-7-Fluoro-4-(5-(Y3-methyloxetan-3-yl)methoxy)pyridin-2-yl)-2,3-dihydro-1H-inden-1-yl)oxy)phenyl)hex-4-ynoic acid

The title compound was synthesized from (S)-3-(4-(((R)-7-fluoro-4-(5-((3-methyloxetan-3-yl)methoxy)pyridin-2-yl)-2,3-dihydro-1H-inden-1-yl)oxy)phenyl)hex-4-ynoic acid methyl ester through the same procedure as used in Example 1. MS ESI (positive) m/z: 516.13 (M+H).

¹H NMR (400 MHz, CDCl₃) δ 8.45 (d, J=2.8 Hz, 1H), 7.65-7.60 (m, 1H), 7.49 (d, J=8.4 Hz, 1H), 7.37 (dd, J=8.4, 2.4 Hz, 1H), 7.32 (d, J=8.8 Hz, 2H), 7.05 (t, J=8.4 Hz, 1H), 6.94 (d, J=8.4 Hz, 2H), 5.93-5.90 (m, 1H), 4.58 (dd, J=58.2, 6.2 Hz, 4H), 4.15 (s, 3H), 4.11-4.02 (m, 1H), 3.37-3.28 (m, 1H), 3.12-3.02 (m, 1H), 2.86-2.69 (m, 2H), 2.44-2.35 (m, 2H), 1.88-1.82 (m, 3H), 1.48 (s, 3H).

### <Example 15> (S)-3-(4-(((R)-7-Fluoro-4-(5-(((R)-tetrahydrofuran-3-yl)oxy)pyridin-3-yl)-2,3-dihydro-1H-inden-1-yl)oxy)phenyl)hex-4-ynoic Acid

The title compound was synthesized from (S)-3-(4-(((R)-7-fluoro-4-(5-(((R)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 502.19 (M+H).

¹H NMR (400 MHz, CDCl₃) δ 8.38-8.15 (br S, 2H), 7.37-7.30 (m, 4H), 7.07 (t, J=8.4 Hz, 1H), 6.91 (d, J=8.4 Hz, 2H), 5.92 (t, J=4.0 Hz, 1H), 5.08-4.99 (m, 1H), 4.14-3.98 (m, 4H), 3.97-3.91 (m, 1H), 3.22-3.13 (m, 1H), 2.90-2.71 (m, 3H), 2.41-2.37 (m, 2H), 2.34-2.23 (m, 1H), 2.21-2.15 (m, 1H), 1.84 (d, J=2.4 Hz, 3H).

### <Example 16> (S)-3-(4-(((R)-7-Fluoro-4-(5-((tetrahydro-2H-pyran-4-yl)oxy)pyridin-3-yl)-2,3-dihydro-1H-inden-1-yl)oxy)phenyl)hex-4-ynoic acid

The title compound was synthesized from (S)-3-(4-(((R)-7-fluoro-4-(5-((tetrahydro-2H-pyran-4-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 516.16 (M+H).

¹H NMR (400 MHz, CDCl₃) δ 8.30 (d, J=2.4 Hz, 1H), 8.24 (s, 1H), 7.35-7.30 (m, 3H), 7.05 (t, J=8.4 Hz, 1H), 6.94 (d, J=8.4 Hz, 2H), 5.92-5.90 (m, 1H), 4.59-4.55 (m, 1H), 4.11-4.06 (m, 1H), 4.02-3.97 (m, 2H), 3.63-3.57 (m, 2H), 3.27-3.19 (m, 1H), 2.89-2.81 (m, 2H), 2.76-2.70 (m, 1H), 2.39-2.34 (m, 2H), 2.08-2.01 (m, 2H), 1.87-1.80 (m, 5H).

### <Example 17> (S)-3-(4-(((R)-4-(5-Chloro-6-((tetrahydro-2H-pyran-4-yl)oxy)pyridin-3-yl)-7-fluoro-2,3-dihydro-1H-inden-1-yl)oxy)phenyl)hex-4-ynoic acid

The title compound was synthesized from (S)-3-(4-(((R)-4-(5-chloro-6-((tetrahydro-2H-pyran-4-yl)oxy)pyridin-3-yl)-7-fluoro-2,3-dihydro-1H-inden-1-yl)oxy)phenyl)hex-4-ynoic acid methyl ester through the same procedure as used in Example 1. MS ESI (positive) m/z: 550.06 (M+H).

¹H NMR (400 MHz, CDCl₃) δ 8.05 (d, J=2.4 Hz, 1H), 7.71 (d, J=2.0 Hz, 1H), 7.34 (d, J=4.8 Hz, 2H), 7.30-7.26 (m, 1H), 7.03 (t, J=8.4 Hz, 1H), 6.95 (d, J=8.4 Hz, 2H), 5.93-5.89 (m, 1H), 5.40-5.32 (m, 1H), 4.10-4.00 (m, 3H), 3.70-3.63 (m, 2H), 3.31-3.20 (m, 1H), 2.91-2.70 (m, 3H), 2.45-2.32 (m, 2H), 2.15-2.06 (m, 2H), 1.95-1.80 (m, 5H).

### <Example 18> (S)-3-(4-(((R)-4-(5-Cyano-6-(((R)-tetrahydrofuran-3-yl)oxy)pyridin-3-yl)-7-fluoro-2,3-dihydro-1H-inden-1-yl)oxy)phenyl)hex-4-ynoic acid

The title compound was synthesized from (S)-3-(4-(((R)-4-(5-cyano-6-(((R)-tetrahydrofuran-3-yl)oxy)pyridin-3-yl)-7-fluoro-2,3-dihydro-1H-inden-1-yl)oxy)phenyl)hex-4-ynoic acid methyl ester through the same procedure as used in Example 1. MS ESI (positive) m/z: 527.04 (M+H).

¹H NMR (400 MHz, CDCl₃) δ 8.37 (d, J=2.4 Hz, 1H), 7.93 (d, J=2.4 Hz, 1H), 7.34 (d, J=8.8 Hz, 2H), 7.30-7.26 (m, 1H), 7.06 (t, J=8.4 Hz, 1H), 6.96 (d, J=8.4 Hz, 2H), 5.93-5.90 (m, 1H), 5.67-5.63 (m, 1H), 4.17-3.92 (m, 5H), 3.31-3.20 (m, 1H), 2.90-2.71 (m, 3H), 2.45-2.25 (m, 4H), 1.84 (d, J=2.4 Hz, 3H).

### <Example 19> (S)-3-(4-(((R)-4-(5-Cyano-6-((tetrahydro-2H-pyran-4-yl)oxy)pyridin-3-yl)-7-fluoro-2,3-dihydro-1H-inden-1-yl)oxy)phenyl)hex-4-ynoic acid

The title compound was synthesized from (S)-3-(4-(((R)-4-(5-cyano-6-((tetrahydro-2H-pyran-4-yl)oxy)pyridin-3-yl)-7-fluoro-2,3-dihydro-1H-inden-1-yl)oxy)phenyl)hex-4-ynoic acid methyl ester through the same procedure as used in Example 1. MS ESI (positive) m/z: 540.93 (M+H).

¹H NMR (400 MHz, CDCl₃) δ 8.36 (d, J=2.4 Hz, 1H), 7.93 (d, J=2.4 Hz, 1H), 7.34 (d, J=8.8 Hz, 2H), 7.29-7.26 (m, 1H), 7.06 (t, J=8.4 Hz, 1H), 6.96 (d, J=8.8 Hz, 2H), 5.93-5.91 (m, 1H), 5.41-5.39 (m, 1H), 4.08-4.01 (m, 3H), 3.68-3.62 (m, 2H), 3.29-3.21 (m, 1H), 2.88-2.80 (m, 2H), 2.76-2.71 (m, 1H), 2.40-2.37 (m, 2H), 3.13-2.09 (m, 2H), 1.93-1.89 (m, 2H), 1.85 (d, J=2.4 Hz, 3H).

### <Example 20> (S)-3-(4-(((R)-5-Cyano-4-(6-(((R)-tetrahydrofuran-3-yl)oxy)pyridin-3-yl)-2,3-dihydro-1H-inden-1-yl)oxy)phenyl)hex-4-ynoic acid

The title compound was synthesized from (S)-3-(4-(((R)-5-cyano-4-(6-(((R)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 509.12 (M+H).

¹H NMR (400 MHz, CDCl₃) δ 8.19 (d, J=2.4 Hz, 1H), 7.89-7.64 (m, 2H), 7.50 (d, J=8.0 Hz, 1H), 7.35 (d, J=8.8 Hz, 2H), 6.95 (d, J=8.4 Hz, 2H), 6.87 (d, J=8.4 Hz, 1H), 5.79 (t, J=5.8 Hz, 1H), 5.65-5.60 (m, 1H), 4.10-3.90 (m, 5H), 3.05-2.97 (m, 1H), 2.90-2.53 (m, 4H), 2.35-2.22 (m, 3H), 1.83 (d, J=2.0 Hz, 3H).

### <Example 21> (S)-3-(4-(((R)-5-Fluoro-4-(6-(((R)-tetrahydrofuran-3-yl)oxy)pyridin-3-yl)-2,3-dihydro-1H-inden-1-yl)oxy)phenyl)hex-4-ynoic acid

The title compound was synthesized from (S)-3-(4-(((R)-5-fluoro-4-(6-(((R)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 502.22 (M+H).

¹H NMR (400 MHz, CDCl₃) δ 8.14 (d, J=2.4 Hz, 1H), 7.64 (d, J=8.6 Hz, 1H), 7.50 (d, J=8.0 Hz, 1H), 7.47 (d, J=8.8 Hz, 2H), 7.08 (t, J=8.4 Hz, 1H), 6.94 (d, J=8.4 Hz, 2H), 6.84 (d, J=4.4 Hz, 1H), 5.79 (t, J=5.8 Hz, 1H), 5.65-5.60 (m, 1H), 4.10-3.90 (m, 5H), 3.05-2.97 (m, 1H), 2.87-2.72 (m, 3H), 2.56-2.53 (m, 1H), 2.35-2.22 (m, 3H), 1.84 (d, J=2.0 Hz, 3H).

### <Example 22> (S)-3-(4-(((R)-5-Methoxy-4-(6-(((R)-tetrahydrofuran-3-yl)oxy)pyridin-3-yl)-2,3-dihydro-1H-inden-1-yl)oxy)phenyl)hex-4-ynoic acid

The title compound was synthesized from (S)-3-(4-(((R)-5-methoxy-4-(6-(((R)-tetrahydrofuran-3-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 514.16 (M+H).

¹H NMR (400 MHz, CDCl₃) δ 8.11 (d, J=2.4 Hz, 1H), 7.59 (dd, J=8.4, 2.4 Hz, 1H), 7.39 (d, J=8.4 Hz, 1H), 7.32 (d, J=8.4 Hz, 2H), 6.94 (d, J=8.4 Hz, 2H), 6.90 (d, J=8.4 Hz, 1H), 6.79 (d, J=8.4 Hz, 1H), 5.75-5.69 (m, 1H), 5.62-5.57 (m, 1H), 4.12-3.96 (m, 4H), 3.95-3.88 (m, 1H), 3.77 (s, 3H), 3.02-2.92 (m, 1H), 2.85-2.67 (m, 3H), 2.51-2.42 (m, 1H), 2.35-2.24 (m, 1H), 2.20-2.15 (m, 2H), 1.84 (d, J=2.4 Hz, 3H).

### <Example 23> (S)-3-(4-(((R)-5-Cyano-4-(6-((tetrahydro-2H-pyran-4-yl)oxy)pyridin-3-yl)-2,3-dihydro-1H-inden-1-yl)oxy)phenyl)hex-4-ynoic acid

The title compound was synthesized from (S)-3-(4-(((R)-5-cyano-4-(6-((tetrahydro-2H-pyran-4-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 523.09 (M+H).

¹H NMR (400 MHz, CDCl₃) δ 8.16 (d, J=2 Hz, 1H), 7.69-7.65 (m, 2H), 7.02 (t, J=8.6 Hz, 1H), 7.35 (d, J=8.4 Hz, 2H), 6.95 (d, J=8.4 Hz, 2H), 6.85 (d, J=8.6 Hz, 1H), 5.80-5.77 (m, 1H), 5.33-5.26 (m, 1H), 4.10-4.06 (m, 1H), 4.03-3.98 (m, 2H), 3.66-3.60 (m, 2H), 3.02-2.95 (m, 1H), 2.88-2.81 (m, 2H), 2.76-2.70 (m. 1H), 2.62-2.54 (m, 1H), 2.23-2.15 (m, 1H), 2.12-2.08 (m, 2H), 1.88-1.79 (m, 5H).

### <Example 24> (S)-3-(4-(((R)-5-Fluoro-4-(6-((tetrahydro-2H-pyran-4-yl)oxy)pyridin-3-yl)-2,3-dihydro-1H-inden-1-yl)oxy)phenyl)hex-4-ynoic acid

The title compound was synthesized from (S)-3-(4-(((R)-5-fluoro-4-(6-((tetrahydro-2H-pyran-4-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 516.17 (M+H).

¹H NMR (400 MHz, CDCl₃) δ 8.12 (d, J=2.4 Hz, 1H), 7.61 (d, J=8.6 Hz, 1H), 7.48 (d, J=8.0 Hz, 1H), 7.45 (d, J=8.8 Hz, 2H), 7.06 (t, J=8.4 Hz, 1H), 6.84 (d, J=8.4 Hz, 2H), 6.74 (d, J=4.4 Hz, 1H), 5.68 (t, J=5.8 Hz, 1H), 5.33-5.26 (m, 1H), 4.08-3.96 (m, 3H), 3.65-3.60 (m, 2H), 3.02-2.95 (m, 1H), 2.86-2.79 (m, 2H), 2.74-2.69 (m. 1H), 2.59-2.53 (m, 1H), 2.21-2.16 (m, 1H), 2.11-2.06 (m, 2H), 1.84-1.75 (m, 5H).

### <Example 25> (S)-3-(4-(((R)-5-Methoxy-4-(6-((tetrahydro-2H-pyran-4-yl)oxy)pyridin-3-yl)-2,3-dihydro-1H-inden-1-yl)oxy)phenyl)hex-4-ynoic acid

The title compound was synthesized from (S)-3-(4-(((R)-5-methoxy-4-(6-((tetrahydro-2H-pyran-4-yl)oxy)pyridin-3-yl)-2,3-dihydro-1H-inden-1-yl)oxy)phenyl)hex-4-ynoic acid methyl ester through the same procedure as used in Example 1. MS ESI (positive) m/z: 528.16 (M+H).

¹H NMR (400 MHz, CDCl₃) δ 8.10 (d, J=2.4 Hz, 1H), 7.62 (dd, J=8.4, 2.4 Hz, 1H), 7.40 (d, J=8.4 Hz, 1H), 7.33 (d, J=8.4 Hz, 2H), 6.95 (d, J=8.4 Hz, 2H), 6.91 (d, J=8.4 Hz, 1H), 6.78 (d, J=8.4 Hz, 1H), 5.75-5.59 (m, 1H), 5.29-5.20 (m, 1H), 4.11-3.98 (m, 3H), 3.76 (s, 3H), 3.68-3.61 (m, 2H), 3.11-2.92 (m, 1H), 2.79-2.62 (m, 3H), 2.50-2.40 (m, 1H), 2.23-2.11 (m, 3H), 1.88-1.78 (m, 5H).

### <Example 26> (S)-3-(4-(((R)-7-Fluoro-4-(6-(((R)-tetrahydrofuran-3-yl)oxy)pyridin-3-yl)-2,3-dihydro-1H-inden-1-yl)amino)phenyl)hex-4-ynoic Acid

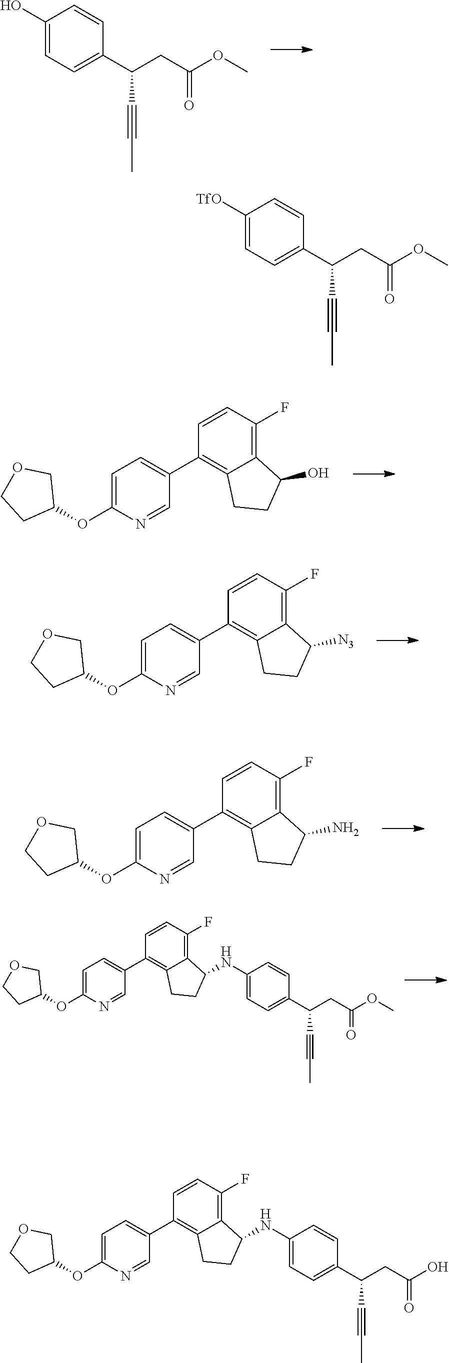

### Step 1: (S)-3-(4-(((Trifluoromethyl)sulfonyl)oxy)phenyl)hex-4-ynoate methyl ester

Trifluoromethanesulfonic anhydride (1.2 eq.) was added to a solution of triethylamine (3.0 eq.) and (3S)-3-(4-Hydroxy-phenyl)-hex-4-ynoic acid methyl ester (1.0 eq.) in dichloromethane (3.5 M). The reaction mixture was stirred at room temperature for 18 h. The mixture was diluted with dichloromethane and washed with saturated aqueous sodium bicarbonate solution. The organic layer was washed with brine and dried over magnesium sulfate, filtered and concentrated. The resultant residue was purified by flash column chromatography on silica gel to afford (S)-3-(4-(((trifluoromethyl)sulfonyl)oxy)phenyl)hex-4-ynoate methyl ester.

### Step 2: 5-((R)-1-Azido-7-fluoro-2,3-dihydro-1H-inden-4-yl)-2-(((R)-tetrahydrofuran-3-yl)oxy)pyridine

Diphenyl phosphoryl azide (1.1 eq.) was added to a solution of (S)-7-fluoro-4-(6-(((R)-tetrahydrofuran-3-yl)oxy)pyridin-3-yl)-2,3-dihydro-1H-inden-1-ol (1.0 eq.) and 1,8-diazabicyclo(5.4.0)undec-7-ene (1.6 eq.) in toluene (0.2 M) at 4° C. The reaction mixture was stirred at room temperature for 18 h. The mixture was diluted with dichloromethane and washed with saturated aqueous sodium bicarbonate solution. The organic layer was washed with brine and dried over magnesium sulfate, filtered and concentrated. The resultant residue was purified by flash column chromatography on silica gel to afford 5-((R)-1-azido-7-fluoro-2,3-dihydro-1H-inden-4-yl)-2-(((R)-tetrahydrofuran-3-yl)oxy)pyridine.

### Step 3: (R)-7-Fluoro-4-(6-(((R)-tetrahydrofuran-3-yl)oxy)pyridin-3-yl)-2,3-dihydro-1H-inden-1-amine

5-((R)-1-Azido-7-fluoro-2,3-dihydro-1H-inden-4-yl)-2-(((R)-tetrahydrofuran-3-yl)oxy)pyridine (1.0 eq.) was added to a suspension of 10% palladium/charcoal (0.6 eq.) in ethanol (0.1 M). The mixture was stirred at room temperature for 2 h under H₂ atmosphere. The reaction mixture is filtered through Celite and concentrated. No further purification was needed to afford (R)-7-fluoro-4-(6-(((R)-tetrahydrofuran-3-yl)oxy)pyridin-3-yl)-2,3-dihydro-1H-inden-1-amine.

### Step 4: (S)-3-(4-(((R)-7-Fluoro-4-(6-(((R)-tetrahydrofuran-3-yl)oxy)pyridin-3-yl)-2,3-dihydro-1H-inden-1-yl)amino)phenyl)hex-4-ynoic acid methyl ester

Tris(dibenzylideneacetone)dipalladium(0)(0.05 eq.) was added to a solution of (R)-7-fluoro-4-(6-(((R)-tetrahydrofuran-3-yl)oxy)pyridin-3-yl)-2,3-dihydro-1H-inden-1-amine (1.0 eq.), (S)-3-(4-(((trifluoromethyl)sulfonyl)oxy)phenyl)hex-4-ynoate methyl ester (1.2 eq.), 2-dicyclohexylphosphino-2′,4″,6′-triisopropylbiphenyl (0.2 eq.), and sodium tert-butoxide (2.5 eq.) in 1,4-dioxane (0.1 M). The reaction mixture was stirred at room temperature for 18 h. Then the mixture was reacted under microwave irradiation for 1 h. The reaction mixture was diluted with ethyl acetate and washed with water. The organic layer was washed with brine and dried over magnesium sulfate, filtered and concentrated. The resultant residue was purified by flash column chromatography on silica gel to afford (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid methyl ester.

### Step 5: (S)-3-(4-(((R)-7-Fluoro-4-(6-(((R)-tetrahydrofuran-3-yl)oxy)pyridin-3-yl)-2,3-dihydro-1H-inden-1-yl)amino)phenyl)hex-4-ynoic acid

2.0 M aqueous lithium hydroxide solution (5.0 eq.) was added to a solution of (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid methyl ester (1.0 eq.) in tetrahydrofuran (1.0 M) and methanol (4.0 M) at 4° C. The reaction mixture was stirred at room temperature for 18 h. The mixture was neutralized with saturated aqueous ammonium chloride solution and diluted with ethyl acetate. The organic layer was washed with brine, dried over magnesium sulfate, filtered, and concentrated. The resultant residue was purified by flash column chromatography on silica gel to afford (S)-3-(4-(((R)-7-fluoro-4-(6-(((R)-tetrahydrofuran-3-yl)oxy)pyridin-3-yl)-2,3-dihydro-1H-inden-1-yl)amino)phenyl)hex-4-ynoic acid. MS ESI (positive) m/z: 501.15 (M+H).

¹H NMR (400 MHz, MeOD) δ 8.21 (d, J=2.0 Hz, 1H), 7.81 (dd, J=8.8, 2.4 Hz, 1H), 7.34-7.28 (m, 1H), 7.17 (d, J=8.4 Hz, 2H), 7.04 (t, J=8.8 Hz, 1H), 6.89 (d, J=8.8 Hz, 1H), 6.70 (d, J=8.4 Hz, 2H), 5.61-5.57 (m, 1H), 5.21-5.18 (m, 1H), 4.64 (br s, 1H), 4.09-3.89 (m, 5H), 3.29-3.20 (m, 1H), 2.91-2.82 (m, 1H), 2.67-2.60 (m, 2H), 2.40-2.28 (m, 2H), 2.22-2.12 (m, 2H), 1.83 (d, J=2.4 Hz, 3H).

### <Example 27>3-(6-(((R)-7-Fluoro-4-(6-(((R)-tetrahydrofuran-3-yl)oxy)pyridin-3-yl)-2,3-dihydro-1H-inden-1-yl)oxy)pyridin-3-yl)hex-4-ynoic Acid

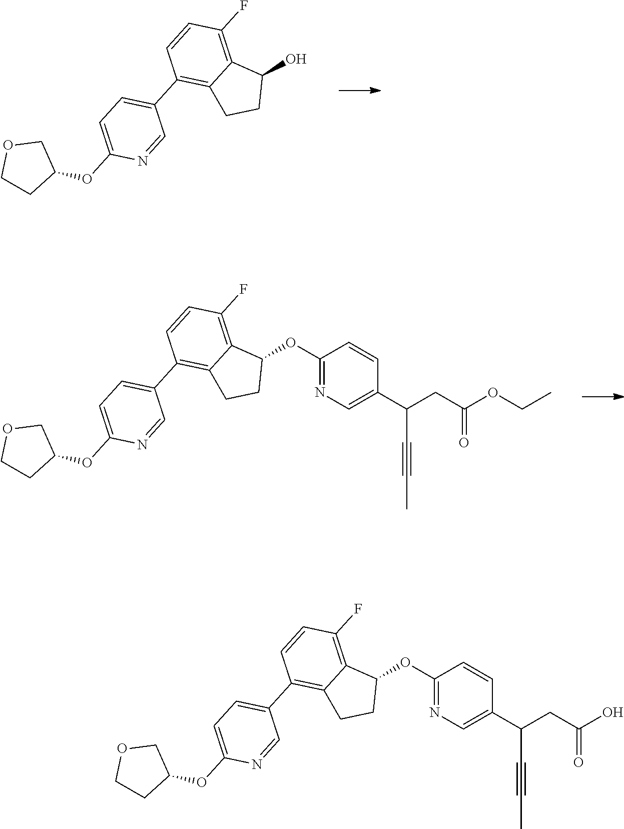 [SMILES: O[C@H]1CCc2c(-c3ccc(O[C@@H]4CCOC4)nc3)ccc(F)c21 ; CC#CC(CC(=O)OCC)c1ccc(O[C@@H]2CCc3c(-c4ccc(O[C@@H]5CCOC5)nc4)ccc(F)c32)nc1 ; CC#CC(CC(=O)O)c1ccc(O[C@@H]2CCc3c(-c4ccc(O[C@@H]5CCOC5)nc4)ccc(F)c32)nc1]

### Step 1: 3-(6-(((R)-7-Fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid ethyl ester

1,1′-(Azodicarbonyl)dipiperidine (1.5 eq.) was added portion wise over 10 min to a solution of (S)-7-fluoro-4-(6-(((R)-tetrahydrofuran-3-yl)oxy)pyridin-3-yl)-2,3-dihydro-1H-inden-1-ol (1.0 eq.), 3-(6-hydroxypyridin-3-yl)hex-4-ynoic acid ethyl ester (1.0 eq.), and tri-n-butylphosphine (1.5 eq.) in toluene (0.1 M) at 4° C. The reaction mixture was stirred at room temperature for 18 h. After addition of hexane (0.05 M) to the reaction mixture, the resulted white solid was removed by filtration. The filtrate was concentrated and then purified by flash column chromatography on silica gel to afford 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid ethyl ester.

### Step 2: 3-(6-(((R)-7-Fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid

2.0 M aqueous lithium hydroxide solution (5.0 eq.) was added to a solution of 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid ethyl ester (1.0 eq.) in tetrahydrofuran (1.0 M) and methanol (4.0 M) at 4° C.°. The reaction mixture was stirred at room temperature for 18 h. The mixture was neutralized with saturated aqueous ammonium chloride solution and diluted with ethyl acetate. The organic layer was washed with brine, dried over magnesium sulfate, filtered, and concentrated. The resultant residue was purified by flash column chromatography on silica gel to afford 3-(6-(((R)-7-fluoro-4-(6-(((R)-tetrahydrofuran-3-yl)oxy)pyridin-3-yl)-2,3-dihydro-1H-inden-1-yl)oxy)pyridin-3-yl)hex-4-ynoic acid. MS ESI (positive) m/z: 503.94 (M+H).

¹H NMR (400 MHz, CDCl₃) δ 8.16 (d, J=2.4 Hz, 1H), 8.12 (d, J=2.6 Hz, 1H), 7.57-7.54 (m, 2H), 7.27-7.25 (m, 1H), 6.94 (t, J=8.6 Hz, 1H), 6.66 (d, J=2.0 Hz, 1H), 6.63 (d, J=2.4, 0.4 Hz, 1H), 6.58-6.54 (m, 1H), 5.52-5.51 (m, 1H), 4.06-3.85 (m, 5H), 3.29-3.19 (m, 1H), 2.79-2.71 (m, 2H), 2.68-2.65 (m, 1H), 2.42-2.32 (m, 1H), 2.29-2.08 (m, 3H), 1.97 (d, J=2.4 Hz, 3H).

### <Comparative Example 1> [(3S)-6-{(2′,6′-Dimethyl-4′-[3-(methylsulfonyl)propoxy]-[1,1′-biphenyl]-3-yl}methoxy)-2,3-dihydro-1-benzofuran-3-yl]acetic Acid

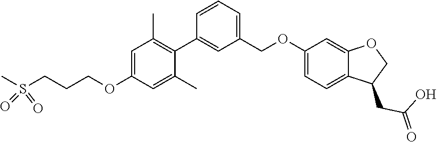 [SMILES: Cc1cc(OCCCS(C)(=O)=O)cc(C)c1-c1cccc(COc2ccc3c(c2)OC[C@H]3CC(=O)O)c1]

[(3S)-6-{(2′,6′-Dimethyl-4′-[3-(methylsulfonyl)propoxy]-[1,1′-biphenyl]-3-yl)}methoxy)-2,3-dihydro-1-benzofuran-3-yl]acetic acid was synthesized based on the reference patent application No. 2008-001931.

[In Vitro Evaluation]

<In Vitro Assay 1> Cell-Based Aequorin Assay

Recombinant cells grown 18 h prior to the test in media without antibiotics were detached by gentle flushing with PBS-EDTA (5 mM EDTA), recovered by centrifugation and resuspended in “assay buffer” (DMEM/HAM's F12 with HEPES+0.1% BSA fatty-acid free). Cells were incubated at room temperature for at least 4 h with Coelenterazine (Molecular Probes). Dose response curves with the reference compounds were performed before testing the compounds.

For agonistic activity testing, 50 μL of cell suspension was injected on 96-well plates with plated 50 μL of test compounds or reference agonist. The resulting emission of light was recorded using the Hamamatsu Functional Drug Screening System 6000 (FDSS 6000).

To standardize the emission of recorded light (determination of the “100% signal”) across plates and across different experiments, some of the wells contained the reference agonist at its EC₁₀₀ obtained during the test validation. Agonistic activities of test compounds were expressed as a percentage of the activity of the reference agonist at its EC₁₀₀ concentration.

TABLE 1

| | Agonistic activity |
|---|---|
| Example No. | (%, 1 μM) |
| 1 | + |
| 3 | + |
| 5 | +++ |
| 6 | +++ |
| 7 | +++ |
| 8 | ++ |
| 15 | ++ |
| 16 | ++ |
| 17 | +++ |
| 18 | +++ |
| 19 | +++ |
| 20 | +++ |
| 21 | +++ |
| 24 | +++ |
| Comparative Example 1 | +++ |

Agonistic activities of compounds in the present invention are shown in Table 1. (+++: over 70, ++: 40-70, +: under 40).

As shown in Table 1, the example compounds of the present invention were confirmed to be excellent in activating GRP40 at 1 μM concentration. In particular, the majority of compounds exhibited more enhanced activities compared to the ‘Comparative Example 1’ which has been known to promote insulin secretion through the activation of GPR40.

### INDUSTRIAL APPLICABILITY

The compounds according to embodiments of the present invention, as GPR40 agonists, are orally available and are extremely effective in lowering blood glucose level to normal state without any risk of inducing hypoglycemia via glucose-dependent insulin secretion. Therefore, compounds and/or therapeutically effective pharmaceutical composition comprising the compounds of the present invention are useful in the treatment, delaying, and/or regression of symptoms of type 2 diabetes.

In addition, compounds disclosed herein modulate glucose excursion via GPR40 activation; the therapeutic effect can also be potentially available in obesity and hypertension.

Furthermore, since the compounds according to embodiments of the present invention have shown improved and/or enhanced therapeutic effects of alleviating and/or treating symptoms of type 2 diabetes compared to pre-existing medications when evaluated of glucose-lowering effects of the compounds on animal models and/or human-organ derived materials, the compounds can be evaluated as being highly useful to potential beneficiaries of the present invention.

1. A process for preparing a compound of the following Formula (I), a racemate thereof, an enantiomer thereof, a diastereomer thereof, or a pharmaceutically acceptable salt of the compound, the racemate, the enantiomer, or the diastereomer: 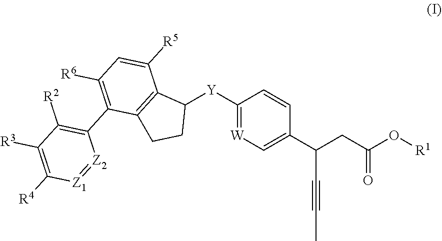
wherein
R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ is hydrogen, halogen, cyano, or C₁₋₄ linear or branched alkoxy when R⁴ is OR⁸; and
R⁴ is hydrogen when R³ is OR⁸;
wherein R⁸ is C₃₋₁₀ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ linear or branched alkoxy;
Y is NH or O;
Z₁ and Z₂ are each independently CR⁷ or N;
wherein when Z₁ is N, Z₂ is CR⁷, and when Z₁ is CR⁷, Z₂ is N; and
W is CR⁷ or N;
wherein R⁷ is hydrogen, halogen, cyano, hydroxyl, C₁₋₄ linear or branched alkyl, or C₁₋₄ linear or branched alkoxy,
said method comprising the steps of reacting a compound of the following formula (7): 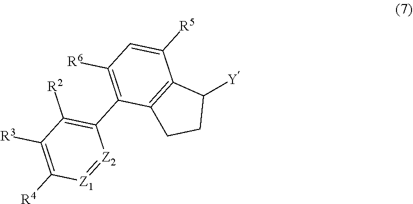
wherein R², R³, R⁴, R⁵, R⁶, Z₁, and Z₂ are as defined above, and
Y′ is NH₂, OH, or tert-butyldimethylsilyloxy,
with a compound of the following formula (8): 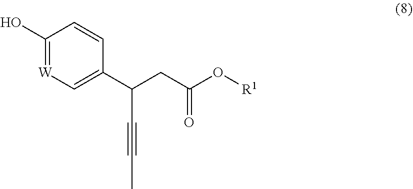
wherein R¹ is as defined above,
to give the compound of Formula (I).

2. The method of claim 1, which further comprises subjecting the compound of formula (I), in which R¹ is a C₁₋₄ linear or branched alkyl, to hydrolysis to give the compound of formula (I) wherein R¹ is hydrogen.

3. The method of claim 1, wherein,
R¹ is hydrogen, or C₁₋₄ linear or branched alkyl;
R² is hydrogen, or C₁₋₄ linear or branched alkyl;
R³ is hydrogen, halogen, cyano, or C₁₋₄ linear alkoxy, when R⁴ is 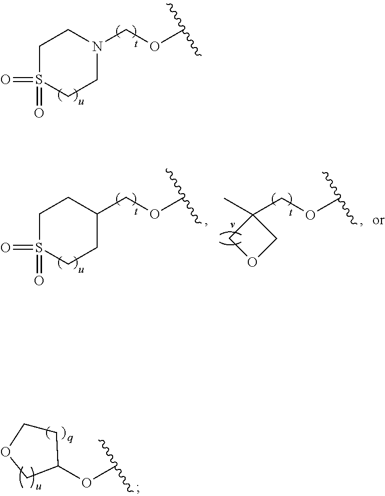
R⁴ is hydrogen, when R³ is 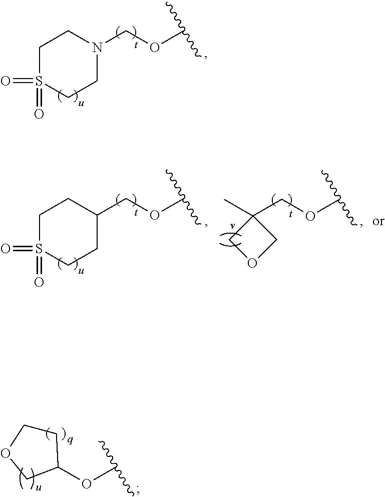
each occurrence of q is independently 0 or 1,
each occurrence of t is independently 0, 1, 2, or 3,
each occurrence of v is independently 1 or 2,
each occurrence of u is independently 1 or 2;
R⁵ and R⁶ are each independently hydrogen, halogen, cyano, or C₁₋₄ linear or branched alkoxy;
Y is NH or O;
Z₁ and Z₂ are each independently CR⁷ or N;
wherein when Z₁ is N, Z₂ is CR⁷, and when Z₁ is CR⁷, Z₂ is N; and
W is CR⁷ or N, wherein R⁷ is hydrogen or C₁₋₂ linear alkyl.

4. The method of claim 1, wherein,
R¹ is hydrogen, or C₁₋₄ linear or branched alkyl;
R² is hydrogen or C₁₋₂ alkyl;
R³ is hydrogen, chloro, cyano or C₁₋₂ alkoxy, when R⁴ is 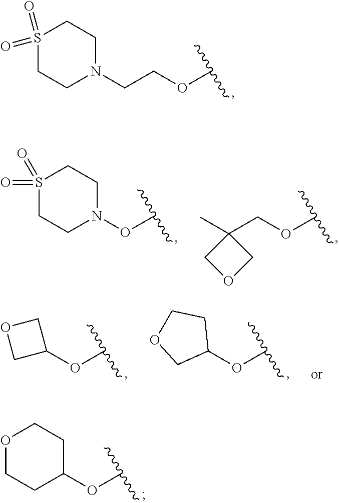
R⁴ is hydrogen when R³ is 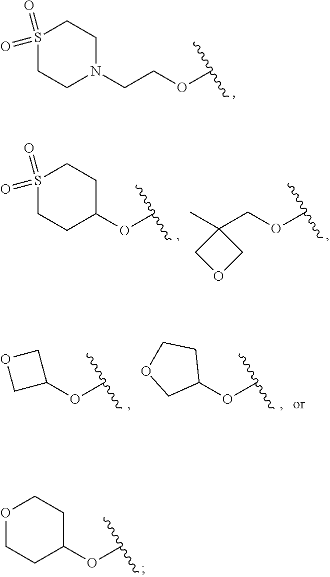
R⁵ and R⁶ are each independently hydrogen, fluoro, cyano, or C₁₋₂ linear alkoxy;
Y is NH or O;
Z₁ and Z₂ are each independently CR⁷ or N, wherein when Z₁ is N, Z₂ is CR⁷ and when Z₁ is CR⁷, Z₂ is N; and
W is CR⁷ or N, wherein R⁷ is hydrogen or C₁₋₂ linear alkyl.

5. The method of claim 1, wherein,
R¹ is hydrogen, or C₁₋₄ linear or branched alkyl;
R² is hydrogen or C₁₋₂ alkyl;
R³ is hydrogen, chloro, cyano or C₁₋₂ alkoxy;
R⁴ is 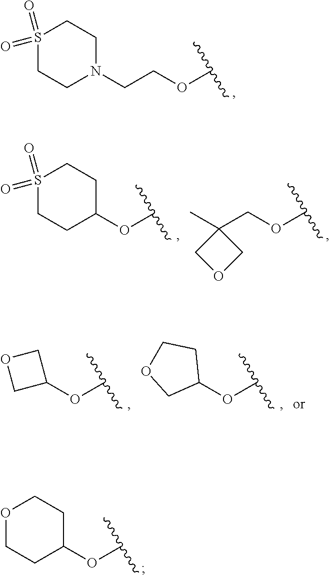
R⁵ and R⁶ are each independently hydrogen, halogen, cyano, or C₁₋₂ linear alkoxy;
Y′ is NH₂ or OH.

6. The method of claim 5, wherein Y is O.

7. The method of claim 5, wherein R⁴ is

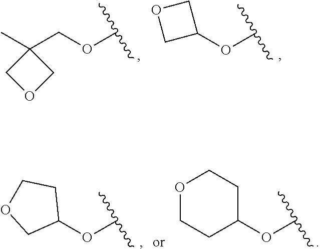

8. The method of claim 5, wherein R⁵ is halogen and R⁶ is hydrogen.

9. The method of claim 8, wherein R⁵ is fluorine.

10. The method of claim 5, wherein R⁵ is hydrogen and R⁶ is halogen, cyano, or a C₁₋₄ linear or branched alkoxy.

11. The method of claim 1, wherein the reaction of the compound of formula (7) and the compound of formula (8) is carried out in the presence of 1,1′-(azodicarbonyl)dipiperidine.

12. A compound of the following formula (7): 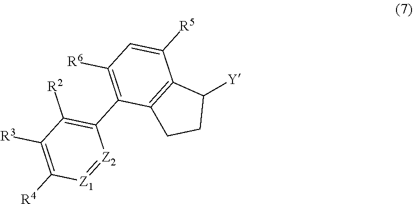
R¹ is hydrogen, or C₁₋₄ linear or branched alkyl;
R² is hydrogen, cyano, hydroxyl, C₁₋₄ linear or branched alkyl, or C₁₋₄ linear or branched alkoxy;
R³ is hydrogen, halogen, cyano, or C₁₋₄ linear or branched alkoxy when R⁴ is OR⁸; and
R⁴ is hydrogen when R³ is OR⁸;
wherein R⁸ is C₃₋₁₀ heterocycloalkyl comprising 1-4 hetero atoms selected from the group consisting of N, O, and S, or alkyl substituted with C₃₋₁₀ heterocycloalkyl comprising 1-4 hetero atoms selected from the group consisting of N, O, and S;
R⁵ and R⁶ are each independently hydrogen, halogen, cyano, halomethyl, hydroxyl, C₁₋₄ linear or branched alkyl, or C₁₋₄ linear or branched alkoxy;
Z₁ and Z₂ are each independently CR⁷ or N;
wherein when Z₁ is N, Z₂ is CR⁷, and when Z₁ is CR⁷, Z₂ is N; and
Y′ is NH₂, OH, or a tert-butyldimethylsilyloxy group.

13. The compound of claim 12, wherein,
R¹ is hydrogen, or C₁₋₄ linear or branched alkyl;
R² is hydrogen, or C₁₋₄ linear or branched alkyl;
R³ is hydrogen, halogen, cyano, or C₁₋₄ linear alkoxy, when R⁴ is 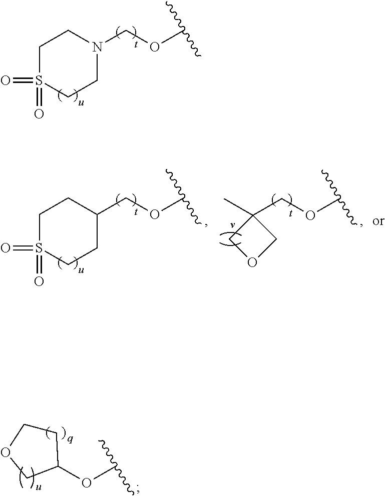
R⁴ is hydrogen, when R³ is 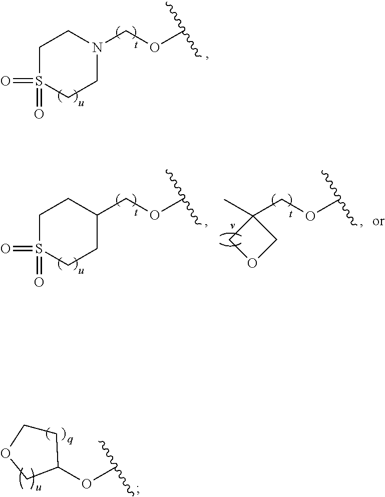
each occurrence of q is independently 0 or 1,
each occurrence of t is independently 0, 1, 2, or 3,
each occurrence of v is independently 1 or 2,
each occurrence of u is independently 1 or 2;
R⁵ and R⁶ are each independently hydrogen, halogen, cyano, or C₁₋₄ linear or branched alkoxy;
Y′ is NH₂ or OH;
Z₁ and Z₂ are each independently CR⁷ or N;
wherein when Z₁ is N, Z₂ is CR⁷, and when Z₁ is CR⁷, Z₂ is N.

14. The compound of claim 12, wherein,
R¹ is hydrogen, or C₁₋₄ linear or branched alkyl;
R² is hydrogen or C₁₋₂ alkyl;
R³ is hydrogen, chloro, cyano or C₁₋₂ alkoxy, when R⁴ is 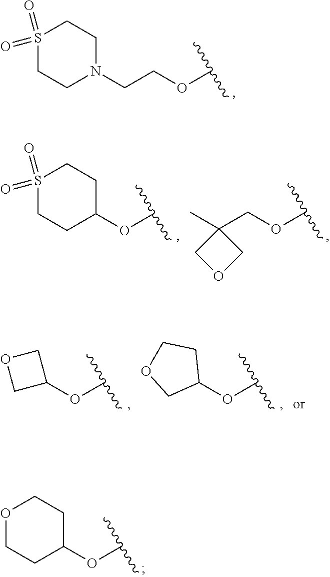
R⁴ is hydrogen when R³ is 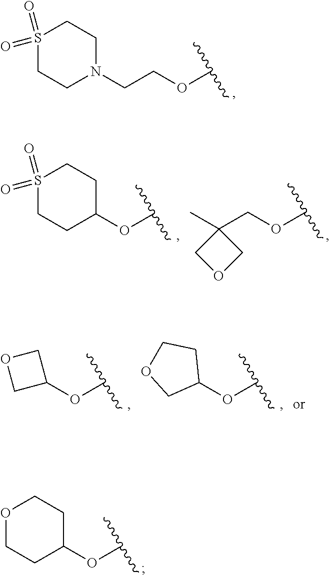
R⁵ and R⁶ are each independently hydrogen, fluoro, cyano, or C₁₋₂ linear alkoxy;
Y′ is NH₂ or OH; and
Z₁ and Z₂ are each independently CR⁷ or N, wherein when Z₁ is N, Z₂ is CR⁷ and when Z₁ is CR⁷, Z₂ is N.

15. The compound of claim 12, wherein,
R¹ is hydrogen, or C₁₋₄ linear or branched alkyl;
R² is hydrogen or C₁₋₂ alkyl;
R³ is hydrogen, chloro, cyano or C₁₋₂ alkoxy;
R⁴ is 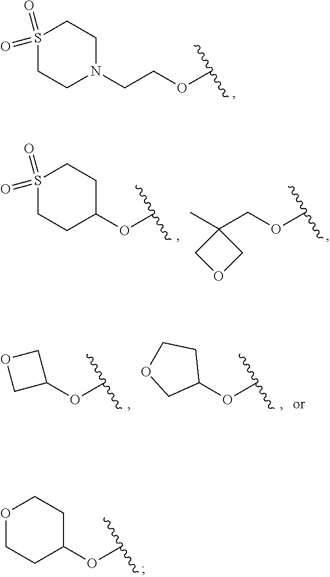
R⁵ and R⁶ are each independently hydrogen, halogen, cyano, or C₁₋₂ linear alkoxy;
Y′ is NH₂ or OH.

16. The compound of claim 15, wherein R⁴ is

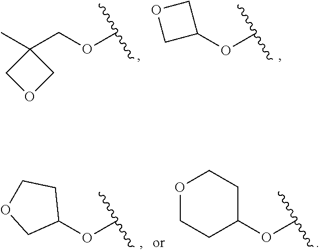

17. The compound of claim 15, wherein R⁵ is halogen and R⁶ is hydrogen.

18. The compound of claim 17, wherein R⁵ is fluorine.

19. The compound of claim 15, wherein R⁵ is hydrogen and R⁶ is halogen, cyano, or a C₁₋₄ linear or branched alkoxy.
